SEQUENCE LISTING (5 sequences)

SEQ ID NO: 1 - DNA, 20 bp, Artificial Sequence
  Description of Artificial Sequence CpG oligonucleotide
tccatgacgttcctgacgtt

SEQ ID NO: 2 - DNA, 20 bp, Artificial Sequence
  Description of Artificial Sequence inactive CpG molecule
tccaggacttctctcaggtt

SEQ ID NO: 3 - DNA, 1914 bp, Artificial Sequence
  Description of Artificial Sequence HCV-1 E1/E2/p7 region
tctttctctatcttccttctggccctgctctcttgcttgactgtgcccgcttcggcctaccaagtgcgcaactccacggggctctaccacgtcaccaatgattgccctaactcgagtattgtgtacgaggcggccgatgccatcctgcacactccggggtgcgtcccttgcgttcgcgagggcaacgcctcgaggtgttgggtggcgatgacccctacggtggccaccagggatggcaaactccccgcgacgcagcttcgacgtcacatcgatctgcttgtcgggagcgccaccctctgttcggccctctacgtgggggacctgtgcgggtctgtctttcttgtcggccaactgtttaccttctctcccaggcgccactggacgacgcaaggttgcaattgctctatctatcccggccatataacgggtcaccgcatggcatgggatatgatgatgaactggtcccctacgacggcgttggtaatggctcagctgctccggatcccacaagccatcttggacatgatcgctggtgctcactggggagtcctggcgggcatagcgtatttctccatggtggggaactgggcgaaggtcctggtagtgctgctgctatttgccggcgtcgacgcggaaacccacgtcaccgggggaagtgccggccacactgtgtctggatttgttagcctcctcgcaccaggcgccaagcagaacgtccagctgatcaacaccaacggcagttggcacctcaatagcacggccctgaactgcaatgatagcctcaacaccggctggttggcagggcttttctatcaccacaagttcaactcttcaggctgtcctgagaggctagccagctgccgaccccttaccgattttgaccagggctggggccctatcagttatgccaacggaagcggccccgaccagcgcccctactgctggcactaccccccaaaaccttgcggtattgtgcccgcgaagagtgtgtgtggtccggtatattgcttcactcccagccccgtggtggtgggaacgaccgacaggtcgggcgcgcccacctacagctggggtgaaaatgatacggacgtcttcgtccttaacaataccaggccaccgctgggcaattggttcggttgtacctggatgaactcaactggattcaccaaagtgtgcggagcgcctccttgtgtcatcggaggggcgggcaacaacaccctgcactgccccactgattgcttccgcaagcatccggacgccacatactctcggtgcggctccggtccctggatcacacccaggtgcctggtcgactacccgtataggctttggcattatccttgtaccatcaactacactatatttaaaatcaggatgtacgtgggaggggtcgagcacaggctggaagctgcctgcaactggacgcggggcgaacgttgcgatctggaagatagggacaggtccgagctcagcccgttactgctgaccactacacagtggcaggtcctcccgtgttccttcacaaccctgccagccttgtccaccggcctcatccacctccaccagaacattgtggacgtgcagtacttgtacggggtggggtcaagcatcgcgtcctgggccattaagtgggagtacgtcgtcctcctgttccttctgcttgcagacgcgcgcgtctgctcctgcttgtggatgatgctactcatatcccaagcggaagcggctttggagaacctcgtaatacttaatgcagcatccctggccgggacgcacggtcttgtatccttcctcgtgttcttctgctttgcatggtatctgaagggtaagtgggtgcccggagcggtctacaccttctacgggatgtggcctctcctcctgctcctgttggcgttgccccagcgggcgtacgcgtaa

SEQ ID NO: 4 - protein, 637 aa, Artificial Sequence
  Description of Artificial Sequence HCV-1 E1/E2/p7 region amino acid
SFSIFLLALLSCLTVPASAYQVRNSTGLYHVTNDCPNSSIVYEAADAILHTPGCVPCVREGNASRCWVAMTPTVATRDGKLPATQLRRHIDLLVGSATLCSALYVGDLCGSVFLVGQLFTFSPRRHWTTQGCNCSIYPGHITGHRMAWDMMMNWSPTTALVMAQLLRIPQAILDMIAGAHWGVLAGIAYFSMVGNWAKVLVVLLLFAGVDAETHVTGGSAGHTVSGFVSLLAPGAKQNVQLINTNGSWHLNSTALNCNDSLNTGWLAGLFYHHKFNSSGCPERLASCRPLTDFDQGWGPISYANGSGPDQRPYCWHYPPKPCGIVPAKSVCGPVYCFTPSPVVVGTTDRSGAPTYSWGENDTDVFVLNNTRPPLGNWFGCTWMNSTGFTKVCGAPPCVIGGAGNNTLHCPTDCFRKHPDATYSRCGSGPWITPRCLVDYPYRLWHYPCTINYTIFKIRMYVGGVEHRLEAACNWTRGERCDLEDRDRSELSPLLLTTTQWQVLPCSFTTLPALSTGLIHLHQNIVDVQYLYGVGSSIASWAIKWEYVVLLFLLLADARVCSCLWMMLLISQAEAALENLVILNAASLAGTHGLVSFLVFFCFAWYLKGKWVPGAVYTFYGMWPLLLLLLALPQRAYA

SEQ ID NO: 5 - DNA, 24 bp, Artificial Sequence
  Description of Artificial Sequence CpG oligonucleotide
tcgtcgttttgtcgttttgtcgtt

(19) United States
(12) Patent Application Publication
(10) Pub. No.: US 2005/0255124 A1
(43) Pub. Date: Nov. 17, 2005

(54) HCV E1E2 vaccine compositions

(75) Inventors: Michael Hougton, Danville, CA (US); Stephen R. Coates, Orinda, CA (US); Derek O'Hagan, Berkeley, CA (US); Yiu-Lian Fong, Lafayette, CA (US)

Correspondence Address: Chiron Corporation;Intellectual Property - R440, P.O. Box 8097, Emeryville, CA, 94662-8097, US

(73) Assignee: Chiron Corporation, Emeryville, CA (US)

(21) Appl. No.: 11/154,324

(22) Filed: Jun. 16, 2005

Related U.S. Application Data
(63) Continuation of application No. 10/187,257, filed on Jun. 28, 2002.
(60) Provisional application No. 60/302,227, filed on Jun. 29, 2001.

(51) Int. Cl. C12Q 1/70; A61K 39/29; A61K 48/00

(52) U.S. Cl. 424/189.1; 514/44

(57) ABSTRACT

HCV E1E2 compositions comprising E1E2 antigens, submicron oil-in-water emulsions and/or immunostimulatory nucleic acid sequences are described. The compositions can be used in methods of stimulating an immune response in a vertebrate subject.

### CROSS-REFERENCES TO RELATED APPLICATIONS

This application is a continuation of U.S. patent application Ser. No. 10/187,257, filed Jun. 28, 2002, which is related to provisional patent application No. 60/302,227, filed Jun. 29, 2001, from which application priority is claimed under 35 USC '119(e)(1), and which applications are incorporated herein by reference in their entirety.

### TECHNICAL FIELD

The present invention pertains generally to vaccine compositions. In particular, the invention relates to HCV E1E2 vaccine compositions comprising E1E2 antigens, submicron oil-in-water emulsions and/or CpG oligonucleotides.

### BACKGROUND OF THE INVENTION

Hepatitis C Virus (HCV) is the principal cause of parenteral non-A, non-B hepatitis (NANBH). The virus is present in 0.4 to 2.0% of blood donors. Chronic hepatitis develops in about 50% of infections and of these, approximately 20% of infected individuals develop liver cirrhosis which sometimes leads to hepatocellular carcinoma. Accordingly, the study and control of the disease is of medical importance.

HCV was first identified and characterized as a cause of NANBH by Houghton et al. The viral genomic sequence of HCV is known, as are methods for obtaining the sequence. See, e.g., International Publication Nos. WO 89/04669; WO 90/11089; and WO 90/14436. HCV has a 9.5 kb positive-sense, single-stranded RNA genome and is a member of the Flaviridae family of viruses. At least six distinct, but related genotypes of HCV, based on phylogenetic analyses, have been identified (Simmonds et al., J. Gen. Virol. (1993) 74:2391-2399). The virus encodes a single polyprotein having more than 3000 amino acid residues (Choo et al., Science (1989) 244:359-362; Choo et al., Proc. Natl. Acad. Sci. USA (1991) 88:2451-2455; Han et al., Proc. Natl. Acad. Sci. USA (1991) 88:2451-2455; Han et al., Proc. Natl. Acad. Sci. USA (1991) 88:1711-1715). The polyprotein is processed co- and post-translationally into both structural and non-structural (NS) proteins.

In particular, as shown in FIG. 1, several proteins are encoded by the HCV genome. The order and nomenclature of the cleavage products of the HCV polyprotein is as follows: NH₂-C-E1-E2-p7-NS2-NS3-NS4a-NS4b-NS5a-NS5b-COOH. Initial cleavage of the polyprotein is catalyzed by host proteases which liberate three structural proteins, the N-terminal nucleocapsid protein (termed “core”) and two envelope glycoproteins, “E1” (also known as E) and “E2” (also known as E2/NS1), as well as nonstructural (NS) proteins that contain the viral enzymes. The NS regions are termed NS2, NS3, NS4 and NS5. NS2 is an integral membrane protein with proteolytic activity and, in combination with NS3, cleaves the NS2-NS3 sissle bond which in turn generates the NS3 N-terminus and releases a large polyprotein that includes both serine protease and RNA helicase activities. The NS3 protease serves to process the remaining polyprotein. In these reactions, NS3 liberates an NS3 cofactor (NS4a), two proteins (NS4b and NS5a), and an RNA-dependent RNA polymerase (NS5b). Completion of polyprotein maturation is initiated by autocatalytic cleavage at the NS3-NS4a junction, catalyzed by the NS3 serine protease.

E1 is detected as a 32-35 kDa species and is converted into a single endo H-sensitive band of approximately 18 kDa. By contrast, E2 displays a complex pattern upon immunoprecipitation consistent with the generation of multiple species (Spaete et al., Virol. (1992) 188:819-830; Selby et al., J. Virol. (1996) 70:5177-5182; Grakoui et al., J. Virol. (1993) 67:1385-1395; Tomei et al., J. Virol. (1993) 67:4017-4026.). The HCV envelope glycoproteins E1 and E2 form a stable complex that is co-immunoprecipitable (Grakoui et al., J. Virol. (1993) 67:1385-1395; Lanford et al., Virology (1993) 197:225-235; Ralston et al., J. Virol. (1993) 67:6753-6761).

E1 and E2 are retained within cells and lack complex carbohydrate when expressed stably or in a transient Vaccinia virus system (Spaete et al., Virology (1992) 188:819-830; Ralston et al., J. Virol. (1993) 67:6753-6761). Since the E1 and E2 proteins are normally membrane-bound in these expression systems, secreted forms have been produced in order to facilitate purification of the proteins. See, e.g., U.S. Pat. No. 6,121,020. Additionally, intracellular production of E1E2 in Hela cells has been described. See, e.g., International Publication No. WO 98/50556.

The HCV E1 and E2 glycoproteins are of considerable interest because they have been shown to be protective against viral challenge in primate studies. (Choo et al., Proc. Natl. Acad. Sci. USA (1994) 91:1294-1298). However, there remains a need for effective vaccine compositions comprising these antigens for the prevention of HCV infection.

Vaccine compositions often include immunological adjuvants to enhance immune responses. For example, Complete Freund's adjuvant (CFA) is a powerful immunostimulatory agent that has been successfully used with many antigens on an experimental basis. CFA includes three components: a mineral oil, an emulsifying agent, and killed mycobacteria, such as Mycobacterium tuberculosis. Aqueous antigen solutions are mixed with these components to create a water-in-oil emulsion. Although effective as an adjuvant, CFA causes severe side-effects, including pain, abscess formation and fever, primarily due to the presence of the mycobacterial component. CFA, therefore, is not used in human and veterinary vaccines.

Muramyl dipeptide (MDP) is the minimal unit of the mycobacterial cell wall complex that generates the adjuvant activity observed with CFA. See, e.g., Ellouz et al., Biochem. Biophys. Res. Commun. (1974) 59:1317. Several synthetic analogs of MDP have been generated that exhibit a wide range of adjuvant potency and side-effects. For a review of these analogs, see, Chedid et al., Prog. Allergy (1978) 25:63. Representative analogs of MDP include threonyl derivatives of MDP (Byars et al., Vaccine (1987) 5:223), n-butyl derivatives of MDP (Chedid et al., Infect. Immun. 35:417), and a lipophilic derivative of a muramyl tripeptide (Gisler et al., in Immunomodulations of Microbial Products and Related Synthetic Compounds (1981) Y. Yamamura and S. Kotani, eds., Excerpta Medica, Amsterdam, p. 167).

One lipophilic derivative of MDP is N-acetylmuramyl-L-alanyl-D-isogluatminyl-L-alanine-2-(1′-2′-dipalmitoyl-sn-glycero-3-huydroxyphosphoryloxy)-ethylamine (MTP-PE). This muramyl tripeptide includes phospholipid tails that allow association of the hydrophobic portion of the molecule with a lipid environment while the muramyl peptide portion associates with the aqueous environment. Thus, the MTP-PE itself is able to act as an emulsifying agent to generate stable oil-in-water emulsions. MTP-PE has been used in an emulsion of 4% squalene with 0.008% Tween™ 80, termed MTP-PE-LO (low oil), to deliver the herpes simplex virus gD antigen with effective results (Sanchez-Pescador et al., J. Immunol. (1988) 141:1720-1727), albeit poor physical stability. Recently, MF59, a safe, highly immunogenic, submicron oil-in-water emulsion which contains 4-5% w/v squalene, 0.5% w/v Tween 80™, 0.5% Span 85™, and optionally, varying amounts of MTP-PE, has been developed for use in vaccine compositions. See, e.g., Ott et al., “MF59—Design and Evaluation of a Safe and Potent Adjuvant for Human Vaccines” in Vaccine Design: The Subunit and Adjuvant Approach (Powell, M. F. and Newman, M. J. eds.) Plenum Press, New York, 1995, pp. 277-296. Choo et al., Proc. Natl. Acad. Sci. USA (1994) 91:1294-1298 and Houghton et al., in Viral Hepatitis and Liver Disease (1997), p. 656, describe the use of HCV E1/E2 complexes with submicron oil-in-water emulsions which include MTP-PE.

Bacterial DNA includes unmethylated CpG dinucleotides that have immunostimulatory effects on peripheral blood mononuclear cells in vitro. Krieg et al., J. Clin. Immunol. (1995) 15:284-292. CpG oligonucleotides have been used to enhance immune responses. See, e.g., U.S. Pat. Nos. 6,207,646; 6,214,806; 6,218,371; and 6,406,705.

Despite the use of such adjuvants, conventional vaccines often fail to provide adequate protection against the targeted pathogen. Accordingly, there is a continuing need for effective vaccine compositions against HCV which include safe and non-toxic adjuvants.

### SUMMARY OF THE INVENTION

The present invention is based in part, on the surprising discovery that the use of HCV E1E2 antigens, in combination with submicron oil-in-water emulsions and oligonucleotides containing immunostimulatory nucleic acid sequences (ISS), such as CpY, CpR and unmethylated CpG motifs (a cytosine followed by guanosine and linked by a phosphate bond), provides for significantly higher antibody titers than those observed without such adjuvants. Alternatively, the compositions herein may be used with ISSs alone, without submicron oil-in-water emulsions, or with submicron oil-in-water emulsions alone that lack MTP-PE, without ISSs. The use of such combinations provides a safe and effective approach for enhancing the immunogenicity of HCV E1E2 antigens.

Accordingly, in one embodiment, the invention is directed to a composition comprising an HCV E1E2 antigen and a submicron oil-in-water emulsion that lacks MTP-PE, wherein the submicron oil-in-water emulsion is capable of increasing the immune response to the HCV E1E2 antigen. The composition may further comprise an ISS, such as an oligonucleotide containing unmethylated CpG motifs (a “CpG oligonucleotide”), which, when present, acts to enhance the immune response to the antigen.

In yet another embodiment, the subject invention is directed to a method of stimulating an immune response in a vertebrate subject which comprises administering to the subject a therapeutically effective amount of an HCV E1E2 antigen and a submicron oil-in-water emulsion that lacks MTP-PE, wherein the submicron oil-in-water emulsion is capable of increasing the immune response to the HCV E1E2 antigen. The subject may also be administered one or more ISSs, such as one or more oligonucleotides containing unmethylated CpG motifs, wherein the ISS is capable of increasing the immune response to the HCV E1E2 antigen. The submicron oil-in-water emulsion may be present in the same composition as the antigen or may be administered in a separate composition. Moreover, if an ISS is present, it may be present in the same composition as the antigen and/or the submicron oil-in-water emulsion, or in a different composition.

In still further embodiments, the invention is directed to a method of making a composition comprising combining a submicron oil-in-water emulsion that lacks MTP-PE with an HCV E1E2 antigen. In certain embodiments, the method further comprises combining an ISS, such as an oligonucleotide containing unmethylated CpG motifs capable of increasing the immune response to the HCV E1E2 antigen, with the E1E2 antigen and the submicron oil-in-water emulsion.

In additional embodiments, the invention is directed to a composition comprising an HCV E1E2 antigen and an ISS, such as a CpG oligonucleotide capable of increasing the immune response to the HCV E1E2 antigen.

In yet another embodiment, the subject invention is directed to a method of stimulating an immune response in a vertebrate subject which comprises administering to the subject a therapeutically effective amount of an HCV E1E2 antigen and an ISS, such as a CpG oligonucleotide, wherein the ISS is capable of increasing the immune response to the HCV E1E2 antigen. The ISS may be present in the same composition as the antigen or may be administered in a separate composition.

In still further embodiments, the invention is directed to a method of making a composition comprising combining an ISS, such as a CpG oligonucleotide, with an HCV E1E2 antigen, wherein the ISS is capable of increasing the immune response to the HCV E1E2 antigen.

The CpG molecule in any of the embodiments above may have the formula 5′-X₁X₂CGX₃X₄, where X₁ and X₂ are a sequence selected from the group consisting of GpT, GpG, GpA, ApA, ApT, ApG, CpT, CpA, CpG, TpA, TpT and TpG, and X₃ and X₄ are selected from the group consisting of TpT, CpT, ApT, ApG, CpG, TpC, ApC, CpC, TpA, ApA, GpT, CpA, and TpG, wherein “p” signifies a phosphate bond. In certain embodiments, the CpG oligonucleotide comprises the sequence GACGTT, GACGTC, GTCGTT or GTCGCT, flanked by several additional nucleotides.

In an additional embodiment, the CpG oligonucleotide for use in the present compositions has the sequence 5′-TCCATGACGTTCCTGACGTT-3′ (SEQ ID NO:1) or the sequence 5′-TCGTCGTTTTGTCGTTTTGTCGTT-3′ (SEQ ID NO:5).

In certain embodiments, the submicron oil-in-water emulsion comprises:
(1) a metabolizable oil, wherein the oil is present in an amount of 0.5% to 20% of the total volume and
(2) an emulsifying agent, wherein the emulsifying agent is 0.01% to 2.5% by weight (w/v), and wherein the oil and the emulsifying agent are present in the form of an oil-in-water emulsion having oil droplets substantially all of which are about 100 nm to less than 1 micron in diameter,
wherein the submicron oil-in-water emulsion is capable of increasing the immune response to the HCV E1E2 antigen.

In other embodiments, the submicron oil-in-water emulsion is as described above and lacks any polyoxypropylene-polyoxyethylene block copolymer, as well as any muramyl peptide.

In additional embodiments, the emulsifying agent comprises a polyoxyethylene sorbitan mono-, di-, or triester and/or a sorbitan mono-, di-, or triester.

In certain embodiments, the oil is present in an amount of 1% to 12%, such as 1% to 4%, of the total volume and the emulsifying agent is 0.01% to 1% by weight (w/v), such as 0.01% to 0.05% by weight (w/v).

In other embodiments described herein, the submicron oil-in-water emulsion comprises 4-5% w/v squalene, 0.25-1.0% w/v Tween 80™ (polyoxyelthylenesorbitan monooleate), and/or 0.25-1.0% Span 85™ (sorbitan trioleate), and optionally, N-acetylmuramyl-L-alanyl-D-isogluatminyl-L-alanine-2-(1′-2′-dipalmitoyl-sn-glycero-3-huydroxyphosphoryloxy)-ethylamine (MTP-PE).

In other embodiments, the submicron oil-in-water emulsion consists essentially of:
(1) 5% by volume of squalene; and
(2) one or more emulsifying agents selected from the group consisting of Tween 80™ (polyoxyelthylenesorbitan monooleate) and Span 85™ (sorbitan trioleate), wherein the total amount of emulsifying agent(s) present is 1% by weight (w/v); wherein the squalene and the emulsifying agent(s) are present in the form of an oil-in-water emulsion having oil droplets substantially all of which are about 100 nm to less than 1 micron in diameter and wherein the composition lacks any polyoxypropylene-polyoxyethylene block copolymer, and further wherein the submicron oil-in-water emulsion is capable of increasing the immune response to the HCV antigen.

In other embodiments, the one or more emulsifying agents are polyoxyelthylenesorbitan monooleate and sorbitan trioleate and the total amount of polyoxyelthylenesorbitan monooleate and sorbitan trioleate present is 1% by weight (w/v).

In certain embodiments, the composition lacks a muramyl peptide.

These and other aspects of the present invention will become evident upon reference to the following detailed description and attached drawings. In addition, various references are set forth herein which describe in more detail certain procedures or compositions, and are therefore incorporated by reference in their entirety.

### BRIEF DESCRIPTION OF THE DRAWINGS

FIG. 1 is a diagrammatic representation of the HCV genome, depicting the various regions of the HCV polyprotein.

FIGS. 2A-2C (SEQ ID NOS:3 and 4) shows the nucleotide and corresponding amino acid sequence for the HCV-1 E1/E2/p7 region. The numbers shown in the figure are relative to the full-length HCV-1 polyprotein. The E1, E2 and p7 regions are shown.

FIG. 3 is a diagram of plasmid pMHE1E2-809, encoding E1E2₈₀₉, a representative E1 E2 protein for use with the present invention.

FIG. 4 shows E1E2₈₀₉ EIA antibody titers from mice immunized with E1E2₈₀₉ plus CpG; E1E2₈₀₉ plus MF59; E1E2₈₀₉ plus CpG and MF59; and E1E2₈₀₉ plus 4XMF59, as described in the examples. Circles indicate individual mouse serum antibody titers. Boxes show the geometric mean antibody titer (GMT) of the group of 10 mice. The error bars are comparison intervals for statistically significant differences as determined by one-way analysis of variance.

### DETAILED DESCRIPTION OF THE INVENTION

The practice of the present invention will employ, unless otherwise indicated, conventional methods of chemistry, biochemistry, recombinant DNA techniques and immunology, within the skill of the art. Such techniques are explained fully in the literature. See, e.g., Fundamental Virology, 2nd Edition, vol. I & II (B. N. Fields and D. M. Knipe, eds.); Handbook of Experimental Immunology, Vols. I-IV (D. M. Weir and C. C. Blackwell eds., Blackwell Scientific Publications); T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.).

All publications, patents and patent applications cited herein, whether supra or infra, are hereby incorporated by reference in their entirety.

It must be noted that, as used in this specification and the appended claims, the singular forms “a”, “an” and “the” include plural referents unless the content clearly dictates otherwise. Thus, for example, reference to “an antigen” includes a mixture of two or more antigens, and the like.

The following amino acid abbreviations are used throughout the text:
Alanine: Ala (A) Arginine: Arg (R)
Asparagine: Asn (N) Aspartic acid: Asp (D)
Cysteine: Cys (C) Glutamine: Gln (O)
Glutamic acid: Glu (E) Glycine: Gly (G)
Histidine: His (H) Isoleucine: Ile (I)
Leucine: Leu (L) Lysine: Lys (K)
Methionine: Met (M) Phenylalanine: Phe (F)
Proline: Pro (P) Serine: Ser (S)
Threonine: Thr (T) Tryptophan: Trp (W)
Tyrosine: Tyr (Y) Valine: Val (V)

I. Definitions

In describing the present invention, the following terms will be employed, and are intended to be defined as indicated below.

The terms “polypeptide” and “protein” refer to a polymer of amino acid residues and are not limited to a minimum length of the product. Thus, peptides, oligopeptides, dimers, multimers, and the like, are included within the definition. Both full-length proteins and fragments thereof are encompassed by the definition. The terms also include postexpression modifications of the polypeptide, for example, glycosylation, acetylation, phosphorylation and the like. Furthermore, for purposes of the present invention, a “polypeptide” refers to a protein which includes modifications, such as deletions, additions and substitutions (generally conservative in nature), to the native sequence, so long as the protein maintains the desired activity. These modifications may be deliberate, as through site-directed mutagenesis, or may be accidental, such as through mutations of hosts which produce the proteins or errors due to PCR amplification.

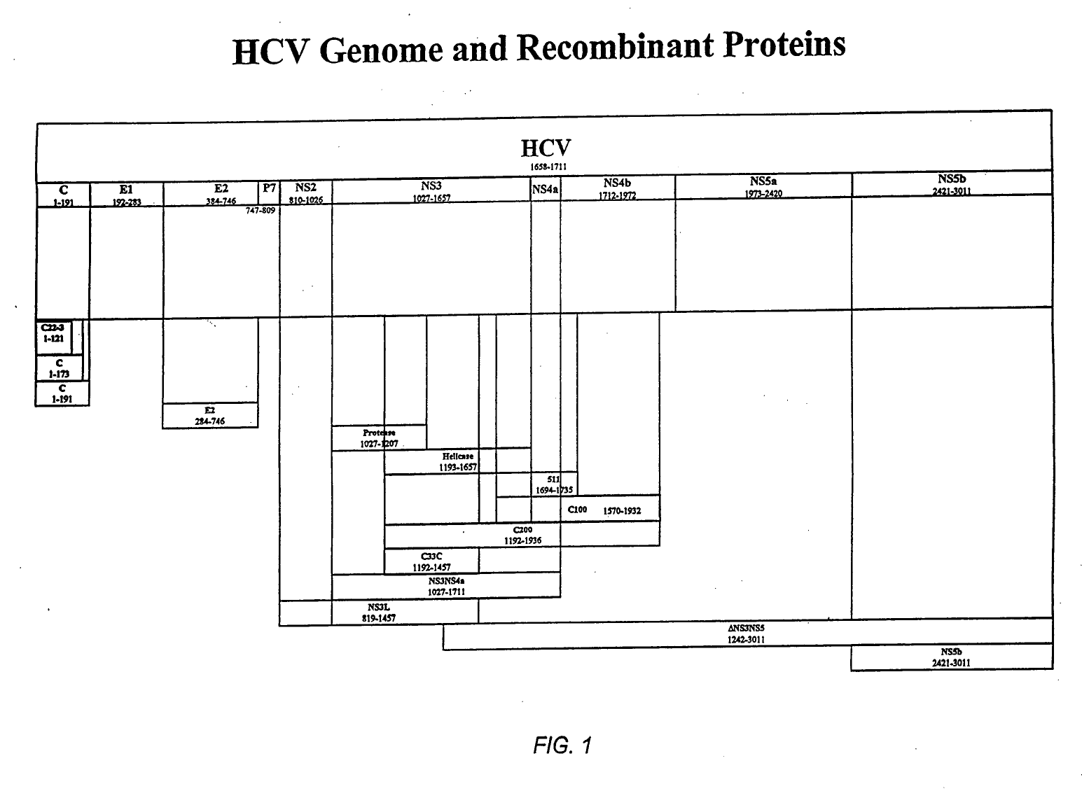

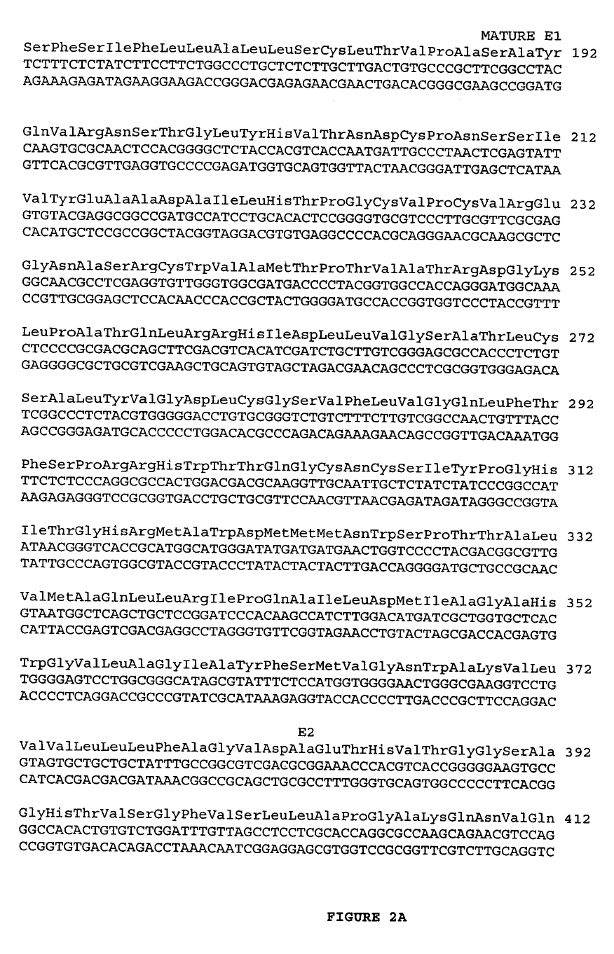

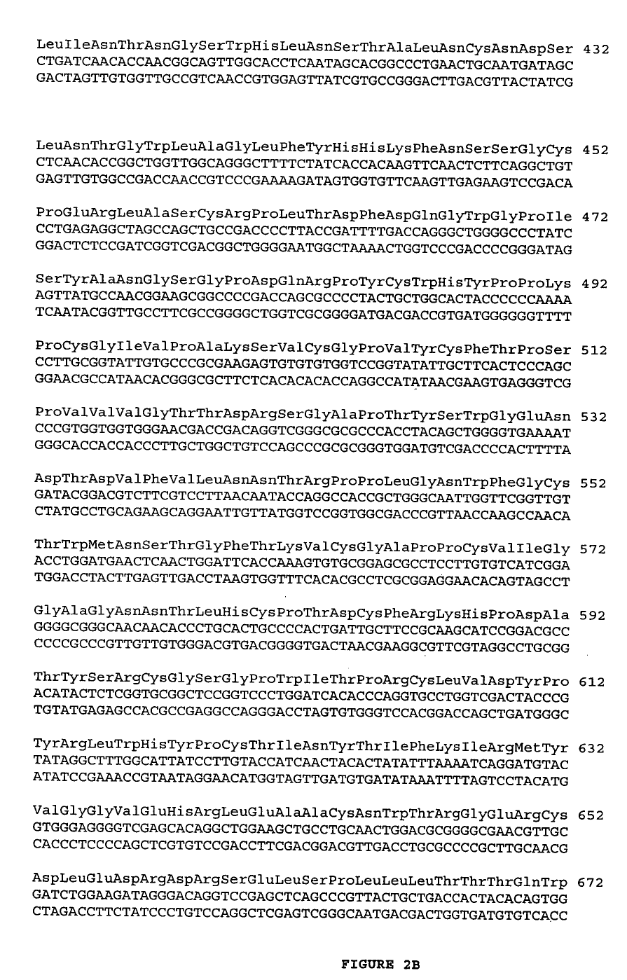

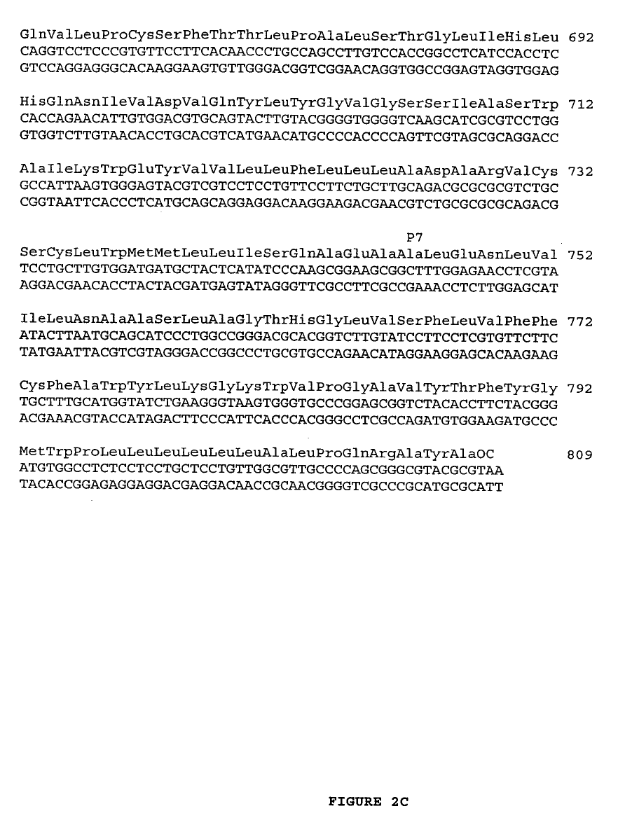

By an “E1 polypeptide” is meant a molecule derived from an HCV E1 region. The mature E1 region of HCV-1 begins at approximately amino acid 192 of the polyprotein and continues to approximately amino acid 383, numbered relative to the full-length HCV-1 polyprotein. (See, FIGS. 1 and 2A-2C. Amino acids 192-383 of FIGS. 2A-2C correspond to amino acid positions 20-211 of SEQ ID NO:4.) Amino acids at around 173 through approximately 191 (amino acids 1-19 of SEQ ID NO: 4) serve as a signal sequence for E1. Thus, by an “E1 polypeptide” is meant either a precursor E1 protein, including the signal sequence, or a mature E1 polypeptide which lacks this sequence, or even an E1 polypeptide with a heterologous signal sequence. The E1 polypeptide includes a C-terminal membrane anchor sequence which occurs at approximately amino acid positions 360-383 (see, International Publication No. WO 96/04301, published Feb. 15, 1996). An E1 polypeptide, as defined herein, may or may not include the C-terminal anchor sequence or portions thereof.

By an “E2 polypeptide” is meant a molecule derived from an HCV E2 region. The mature E2 region of HCV-1 begins at approximately amino acid 383-385, numbered relative to the full-length HCV-1 polyprotein. (See, FIGS. 1 and 2A-2C. Amino acids 383-385 of FIGS. 2A-2C correspond to amino acid positions 211-213 of SEQ ID NO:4.) A signal peptide begins at approximately amino acid 364 of the polyprotein. Thus, by an “E2 polypeptide” is meant either a precursor E2 protein, including the signal sequence, or a mature E2 polypeptide which lacks this sequence, or even an E2 polypeptide with a heterologous signal sequence. The E2 polypeptide includes a C-terminal membrane anchor sequence which occurs at approximately amino acid positions 715-730 and may extend as far as approximately amino acid residue 746 (see, Lin et al., J. Virol. (1994) 68:5063-5073). An E2 polypeptide, as defined herein, may or may not include the C-terminal anchor sequence or portions thereof. Moreover, an E2 polypeptide may also include all or a portion of the p7 region which occurs immediately adjacent to the C-terminus of E2. As shown in FIGS. 1 and 2A-2C, the p7 region is found at positions 747-809, numbered relative to the full-length HCV-1 polyprotein (amino acid positions 575-637 of SEQ ID NO:4). Additionally, it is known that multiple species of HCV E2 exist (Spaete et al., Virol. (1992) 188:819-830; Selby et al., J. Virol. (1996) 70:5177-5182; Grakoui et al., J. Virol. (1993) 67:1385-1395; Tomei et al., J. Virol. (1993) 67:4017-4026). Accordingly, for purposes of the present invention, the term “E2” encompasses any of these species of E2 including, without limitation, species that have deletions of 1-20 or more of the amino acids from the N-terminus of the E2, such as, e.g, deletions of 1, 2, 3, 4, 5 . . . 10 . . . 15, 16, 17, 18, 19 . . . etc. amino acids. Such E2 species include those beginning at amino acid 387, amino acid 402, amino acid 403, etc.

Representative E1 and E2 regions from HCV-1 are shown in FIGS. 2A-2C and SEQ ID NO:4. For purposes of the present invention, the E1 and E2 regions are defined with respect to the amino acid number of the polyprotein encoded by the genome of HCV-1, with the initiator methionine being designated position 1. See, e.g., Choo et al., Proc. Natl. Acad. Sci. USA (1991) 88:2451-2455. However, it should be noted that the term an “E1 polypeptide” or an “E2 polypeptide” as used herein is not limited to the HCV-1 sequence. In this regard, the corresponding E1 or E2 regions in other HCV isolates can be readily determined by aligning sequences from the isolates in a manner that brings the sequences into maximum alignment. This can be performed with any of a number of computer software packages, such as ALIGN 1.0, available from the University of Virginia, Department of Biochemistry (Attn: Dr. William R. Pearson). See, Pearson et al., Proc. Natl. Acad. Sci. USA (1988) 85:2444-2448.

Furthermore, an “E1 polypeptide” or an “E2 polypeptide” as defined herein is not limited to a polypeptide having the exact sequence depicted in the Figures. Indeed, the HCV genome is in a state of constant flux in vivo and contains several variable domains which exhibit relatively high degrees of variability between isolates. A number of conserved and variable regions are known between these strains and, in general, the amino acid sequences of epitopes derived from these regions will have a high degree of sequence homology, e.g., amino acid sequence homology of more than 30%, preferably more than 40%, more than 60%, and even more than 80-90% homology, when the two sequences are aligned. It is readily apparent that the terms encompass E1 and E2 polypeptides from any of the various HCV strains and isolates including isolates having any of the 6 genotypes of HCV described in Simmonds et al., J. Gen. Virol. (1993) 74:2391-2399 (e.g., strains 1, 2, 3, 4 etc.), as well as newly identified isolates, and subtypes of these isolates, such as HCV1a, HCV1b etc.

Thus, for example, the term “E1” or “E2” polypeptide refers to native E1 or E2 sequences from any of the various HCV strains, as well as analogs, muteins and immunogenic fragments, as defined further below. The complete genotypes of many of these strains are known. See, e.g., U.S. Pat. No. 6,150,087 and GenBank Accession Nos. AJ238800 and AJ238799.

Additionally, the terms “E1 polypeptide” and “E2 polypeptide” encompass proteins which include modifications to the native sequence, such as internal deletions, additions and substitutions (generally conservative in nature). These modifications may be deliberate, as through site-directed mutagenesis, or may be accidental, such as through naturally occurring mutational events. All of these modifications are encompassed in the present invention so long as the modified E1 and E2 polypeptides function for their intended purpose. Thus, for example, if the E1 and/or E2 polypeptides are to be used in vaccine compositions, the modifications must be such that immunological activity (i.e., the ability to elicit a humoral or cellular immune response to the polypeptide) is not lost.

By “E1E2” complex is meant a protein containing at least one E1 polypeptide and at least one E2 polypeptide, as described above. Such a complex may also include all or a portion of the p7 region which occurs immediately adjacent to the C-terminus of E2. As shown in FIGS. 1 and 2A-2C, the p7 region is found at positions 747-809, numbered relative to the full-length HCV-1 polyprotein (amino acid positions 575-637 of SEQ ID NO:4). A representative E1E2 complex which includes the p7 protein is termed “E1E2₈₀₉” herein.

The mode of association of E1 and E2 in an E1E2 complex is immaterial. The E1 and E2 polypeptides may be associated through non-covalent interactions such as through electrostatic forces, or by covalent bonds. For example, the E1E2 polypeptides of the present application may be in the form of a fusion protein which includes an immunogenic E1 polypeptide and an immunogenic E2 polypeptide, as defined above. The fusion may be expressed from a polynucleotide encoding an E1E2 chimera. Alternatively, E1E2 complexes may form spontaneously simply by mixing E1 and E2 proteins which have been produced individually. Similarly, when co-expressed and secreted into media, the E1 and E2 proteins can form a complex spontaneously. Thus, the term encompasses E1E2 complexes (also called aggregates) that spontaneously form upon purification of E1 and/or E2. Such aggregates may include one or more E1 monomers in association with one or more E2 monomers. The number of E1 and E2 monomers present need not be equal so long as at least one E1 monomer and one E2 monomer are present. Detection of the presence of an E1E2 complex is readily determined using standard protein detection techniques such as polyacrylamide gel electrophoresis and immunological techniques such as immunoprecipitation.

The terms “analog” and “mutein” refer to biologically active derivatives of the reference molecule, or fragments of such derivatives, that retain desired activity, such as immunoreactivity in assays described herein. In general, the term “analog” refers to compounds having a native polypeptide sequence and structure with one or more amino acid additions, substitutions (generally conservative in nature) and/or deletions, relative to the native molecule, so long as the modifications do not destroy immunogenic activity. The term “mutein” refers to peptides having one or more peptide mimics (“peptoids”), such as those described in International Publication No. WO 91/04282. Preferably, the analog or mutein has at least the same immunoactivity as the native molecule. Methods for making polypeptide analogs and muteins are known in the art and are described further below.

Particularly preferred analogs include substitutions that are conservative in nature, i.e., those substitutions that take place within a family of amino acids that are related in their side chains. Specifically, amino acids are generally divided into four families: (1) acidic—aspartate and glutamate; (2) basic—lysine, arginine, histidine; (3) non-polar—alanine, valine, leucine, isoleucine, proline, phenylalanine, methionine, tryptophan; and (4) uncharged polar—glycine, asparagine, glutamine, cysteine, serine threonine, tyrosine. Phenylalanine, tryptophan, and tyrosine are sometimes classified as aromatic amino acids. For example, it is reasonably predictable that an isolated replacement of leucine with isoleucine or valine, an aspartate with a glutamate, a threonine with a serine, or a similar conservative replacement of an amino acid with a structurally related amino acid, will not have a major effect on the biological activity. For example, the polypeptide of interest may include up to about 5-10 conservative or non-conservative amino acid substitutions, or even up to about 15-25 or 50 conservative or non-conservative amino acid substitutions, or any integer between 5-50, so long as the desired function of the molecule remains intact. One of skill in the art may readily determine regions of the molecule of interest that can tolerate change by reference to Hopp/Woods and Kyte-Doolittle plots, well known in the art.

By “fragment” is intended a polypeptide consisting of only a part of the intact full-length polypeptide sequence and structure. The fragment can include a C-terminal deletion an N-terminal deletion, and/or an internal deletion of the native polypeptide. An “immunogenic fragment” of a particular HCV protein will generally include at least about 5-10 contiguous amino acid residues of the full-length molecule, preferably at least about 15-25 contiguous amino acid residues of the full-length molecule, and most preferably at least about 20-50 or more contiguous amino acid residues of the full-length molecule, that define an epitope, or any integer between 5 amino acids and the full-length sequence, provided that the fragment in question retains the ability to elicit an immunological response as defined herein. For a description of known immunogenic fragments of HCV E1 and E2, see, e.g., Chien et al., International Publication No. WO 93/00365.

The term “epitope” as used herein refers to a sequence of at least about 3 to 5, preferably about 5 to 10 or 15, and not more than about 500 amino acids (or any integer therebetween), which define a sequence that by itself or as part of a larger sequence, elicits an immunological response in the subject to which it is administered. Often, an epitope will bind to an antibody generated in response to such sequence. There is no critical upper limit to the length of the fragment, which may comprise nearly the full-length of the protein sequence, or even a fusion protein comprising two or more epitopes from the HCV polyprotein. An epitope for use in the subject invention is not limited to a polypeptide having the exact sequence of the portion of the parent protein from which it is derived. Indeed, viral genomes are in a state of constant flux and contain several variable domains which exhibit relatively high degrees of variability between isolates. Thus the term “epitope” encompasses sequences identical to the native sequence, as well as modifications to the native sequence, such as deletions, additions and substitutions (generally conservative in nature).

Regions of a given polypeptide that include an epitope can be identified using any number of epitope mapping techniques, well known in the art. See, e.g., Epitope Mapping Protocols in Methods in Molecular Biology, Vol. 66 (Glenn E. Morris, Ed., 1996) Humana Press, Totowa, N.J. For example, linear epitopes may be determined by e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al. (1984) Proc. Natl. Acad. Sci. USA 81:3998-4002; Geysen et al. (1985) Proc. Natl. Acad. Sci. USA 82:178-182; Geysen et al. (1986) Molec. Immunol. 23:709-715, all incorporated herein by reference in their entireties. Using such techniques, a number of epitopes of HCV have been identified. See, e.g., Chien et al., Viral Hepatitis and Liver Disease (1994) pp. 320-324, and further below. Similarly, conformational epitopes are readily identified by determining spatial conformation of amino acids such as by, e.g., x-ray crystallography and 2-dimensional nuclear magnetic resonance. See, e.g., Epitope Mapping Protocols, supra. Antigenic regions of proteins can also be identified using standard antigenicity and hydropathy plots, such as those calculated using, e.g., the Omiga version 1.0 software program available from the Oxford Molecular Group. This computer program employs the Hopp/Woods method, Hopp et al., Proc. Natl. Acad. Sci USA (1981) 78:3824-3828 for determining antigenicity profiles, and the Kyte-Doolittle technique, Kyte et al., J. Mol. Biol. (1982) 157:105-132 for hydropathy plots.

As used herein, the term “conformational epitope” refers to a portion of a full-length protein, or an analog or mutein thereof, having structural features native to the amino acid sequence encoding the epitope within the full-length natural protein. Native structural features include, but are not limited to, glycosylation and three dimensional structure. The length of the epitope defining sequence can be subject to wide variations as these epitopes are believed to be formed by the three-dimensional shape of the antigen (e.g., folding). Thus, amino acids defining the epitope can be relatively few in number, but widely dispersed along the length of the molecule (or even on different molecules in the case of dimers, etc.), being brought into correct epitope conformation via folding. The portions of the antigen between the residues defining the epitope may not be critical to the conformational structure of the epitope. For example, deletion or substitution of these intervening sequences may not affect the conformational epitope provided sequences critical to epitope conformation are maintained (e.g., cysteines involved in disulfide bonding, glycosylation sites, etc.).

Conformational epitopes are readily identified using methods discussed above. Moreover, the presence or absence of a conformational epitope in a given polypeptide can be readily determined through screening the antigen of interest with an antibody (polyclonal serum or monoclonal to the conformational epitope) and comparing its reactivity to that of a denatured version of the antigen which retains only linear epitopes (if any). In such screening using polyclonal antibodies, it may be advantageous to absorb the polyclonal serum first with the denatured antigen and see if it retains antibodies to the antigen of interest. Conformational epitopes derived from the E1 and E2 regions are described in, e.g., International Publication No. WO 94/01778.

An “immunological response” to an HCV antigen or composition is the development in a subject of a humoral and/or a cellular immune response to molecules present in the composition of interest. For purposes of the present invention, a “humoral immune response” refers to an immune response mediated by antibody molecules, while a “cellular immune response” is one mediated by T-lymphocytes and/or other white blood cells. One important aspect of cellular immunity involves an antigen-specific response by cytolytic T-cells (“CTLs”). CTLs have specificity for peptide antigens that are presented in association with proteins encoded by the major histocompatibility complex (MHC) and expressed on the surfaces of cells. CTLs help induce and promote the intracellular destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A “cellular immune response” also refers to the production of cytokines, chemokines and other such molecules produced by activated T-cells and/or other white blood cells, including those derived from CD4+ and CD8+ T-cells. A composition or vaccine that elicits a cellular immune response may serve to sensitize a vertebrate subject by the presentation of antigen in association with MHC molecules at the cell surface. The cell-mediated immune response is directed at, or near, cells presenting antigen at their surface. In addition, antigen-specific T-lymphocytes can be generated to allow for the future protection of an immunized host. The ability of a particular antigen to stimulate a cell-mediated immunological response may be determined by a number of assays, such as by lymphoproliferation (lymphocyte activation) assays, CTL cytotoxic cell assays, or by assaying for T-lymphocytes specific for the antigen in a sensitized subject. Such assays are well known in the art. See, e.g., Erickson et al., J. Immunol. (1993) 151:4189-4199; Doe et al., Eur. J. Immunol. (1994) 24:2369-2376.

Thus, an immunological response as used herein may be one which stimulates the production of CTLs, and/or the production or activation of helper T-cells. The antigen of interest may also elicit an antibody-mediated immune response, including, or example, neutralization of binding (NOB) antibodies. The presence of an NOB antibody response is readily determined by the techniques described in, e.g., Rosa et al., Proc. Natl. Acad. Sci. USA (1996) 93:1759. Hence, an immunological response may include one or more of the following effects: the production of antibodies by B-cells; and/or the activation of suppressor T-cells and/or 8 T-cells directed specifically to an antigen or antigens present in the composition or vaccine of interest. These responses may serve to neutralize infectivity, and/or mediate antibody-complement, or antibody dependent cell cytotoxicity (ADCC) to provide protection or alleviation of symptoms to an immunized host. Such responses can be determined using standard immunoassays and neutralization assays, well known in the art.

As used herein an “immunostimulatory nucleotide sequence” or “ISS” means a polynucleotide that includes at least one immunostimulatory oligonucleotide (ISS-ODN) moiety. The ISS moiety is a single- or double-stranded DNA or RNA oligonucleotide having at least six nucleotide bases that may include, or consist of, a modified oligonucleotide or a sequence of modified nucleosides. The ISS moieties comprise, or may be flanked by, a CG-containing nucleotide sequence or a p(1C) nucleotide sequence, which may be palindromic. The cysteine may be methylated or unmethylated. Examples of particular ISS molecules for use in the present invention include CpG molecules, discussed further below, as well as CpY and CpR molecules and the like.

A component of an HCV E1E2 composition, such as a submicron oil-in-water emulsion or CpG oligonucleotide, enhances the immune response to the HCV E1E2 antigen present in the composition when the composition possesses a greater capacity to elicit an immune response than the immune response elicited by an equivalent amount of the antigen when delivered without the additional component. Such enhanced immunogenicity can be determined by administering the antigen composition with and without the additional components, and comparing antibody titers against the two using standard assays such as radioimmunoassay and ELISAs, well known in the art.

A “recombinant” protein is a protein which retains the desired activity and which has been prepared by recombinant DNA techniques as described herein. In general, the gene of interest is cloned and then expressed in transformed organisms, as described further below. The host organism expresses the foreign gene to produce the protein under expression conditions.

By “isolated” is meant, when referring to a polypeptide, that the indicated molecule is separate and discrete from the whole organism with which the molecule is found in nature or is present in the substantial absence of other biological macro-molecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

By “equivalent antigenic determinant” is meant an antigenic determinant from different sub-species or strains of HCV, such as from strains 1, 2, 3, etc., of HCV which antigenic determinants are not necessarily identical due to sequence variation, but which occur in equivalent positions in the HCV sequence in question. In general the amino acid sequences of equivalent antigenic determinants will have a high degree of sequence homology, e.g., amino acid sequence homology of more than 30%, usually more than 40%, such as more than 60%, and even more than 80-90% homology, when the two sequences are aligned.

“Homology” refers to the percent identity between two polynucleotide or two polypeptide moieties. Two DNA, or two polypeptide sequences are “substantially homologous” to each other when the sequences exhibit at least about 50%, preferably at least about 75%, more preferably at least about 80%-85%, preferably at least about 90%, and most preferably at least about 95%-98% sequence identity over a defined length of the molecules. As used herein, substantially homologous also refers to sequences showing complete identity to the specified DNA or polypeptide sequence.

In general, “identity” refers to an exact nucleotide-to-nucleotide or amino acid-to-amino acid correspondence of two polynucleotides or polypeptide sequences, respectively. Percent identity can be determined by a direct comparison of the sequence information between two molecules by aligning the sequences, counting the exact number of matches between the two aligned sequences, dividing by the length of the shorter sequence, and multiplying the result by 100. Readily available computer programs can be used to aid in the analysis, such as ALIGN, Dayhoff, M. O. in Atlas of Protein Sequence and Structure M. O. Dayhoff ed., 5 Suppl. 3:353-358, National biomedical Research Foundation, Washington, D.C., which adapts the local homology algorithm of Smith and Waterman Advances in Appl. Math. 2:482-489, 1981 for peptide analysis. Programs for determining nucleotide sequence identity are available in the Wisconsin Sequence Analysis Package, Version 8 (available from Genetics Computer Group, Madison, Wis.) for example, the BESTFIT, FASTA and GAP programs, which also rely on the Smith and Waterman algorithm. These programs are readily utilized with the default parameters recommended by the manufacturer and described in the Wisconsin Sequence Analysis Package referred to above. For example, percent identity of a particular nucleotide sequence to a reference sequence can be determined using the homology algorithm of Smith and Waterman with a default scoring table and a gap penalty of six nucleotide positions.

Another method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by ═HIGH SCORE; Databases=non-redundant, GenBank+EMBL+DDBJ+PDB+GenBank CDS translations+Swiss protein+Spupdate+PIR. Details of these programs can be found at the following internet address: http://www.ncbi.nlm.gov/cgi-bin/BLAST.

Alternatively, homology can be determined by hybridization of polynucleotides under conditions which form stable duplexes between homologous regions, followed by digestion with single-stranded-specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; DNA Cloning, supra; Nucleic Acid Hybridization, supra.

II. Modes of Carrying out the Invention

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of compositions and methods similar or equivalent to those described herein can be used in the practice of the present invention, the preferred materials and methods are described herein.

As noted above, the present invention is based on the discovery that HCV E1E2 antigens, in combination with submicron oil-in-water emulsions lacking MTP-PE, as well as with submicron oil-in-water emulsions and immunostimulatory nucleic acid molecules, such as CpG oligonucleotides, provide compositions that elicit significantly higher antibody titers than those observed without such adjuvants. Elicitation of HCV-specific antibodies by E1E2 polypeptides provides both in vitro and in vivo model systems for the development of HCV vaccines, particularly for identifying HCV E1, E2 and HCV E1E2 polypeptide epitopes associated with the production of strong anti-E1, anti-E2 and/or anti E1E2 antibody titers, and/or cellular immune responses directed against HCV. E1E2 polypeptides can also be used to generate an immune response against an HCV in a mammal, particularly an anti-E1, anti-E2 and/or anti-E1E2 antibody response and/or a cellular immune response, for either therapeutic or prophylactic purposes.

In order to further an understanding of the invention, a more detailed discussion is provided below regarding E1E2 polypeptides for use in the subject compositions, as well as production of submicron oil-in-water emulsions, immunostimulatory nucleic acid molecules and compositions comprising the above.

E1E2 Polypeptides

As explained above, the E1E2 complexes for use with the present compositions comprise E1 and E2 polypeptides, associated either through non-covalent or covalent interactions. The genome of the hepatitis C virus typically contains a single open reading frame of approximately 9,600 nucleotides, which is transcribed into a polyprotein. An HCV polyprotein is cleaved to produce a number of distinct products, in the order of NH₂-C-E1-E2-p7-NS2-NS3-NS4a-NS4b-NS5a-NS5b-COOH (see, FIG. 1). The HCV E1 polypeptide is a glycoprotein and extends from approximately amino acid 192 to amino acid 383 (numbered relative to the polyprotein of HCV-1). See, Choo et al., Proc. Natl. Acad. Sci. USA (1991) 88:2451-2455. Amino acids at around 173 through approximately 191 represent a signal sequence for E1. An HCV E2 polypeptide is also a glycoprotein and extends from approximately amino acid 383 or 384 to amino acid 746. A signal peptide for E2 begins at approximately amino acid 364 of the polyprotein. Thus, the term “full-length” E1 or “not truncated” E1 as used herein refers to polypeptides that include, at least, amino acids 192-383 of an HCV polyprotein (numbered relative to HCV-1). With respect to E2, the term “full-length” or “not truncated” as used herein refers to polypeptides that include, at least, amino acids 383 or 384 to amino acid 746 of an HCV polyprotein (numbered relative to HCV-1). As will be evident from this disclosure, E2 polypeptides for use with the present invention may include additional amino acids from the p7 region, such as amino acids 747-809.

As explained above, E2 exists as multiple species (Spaete et al., Virol. (1992) 188:819-830; Selby et al., J. Virol. (1996) 70:5177-5182; Grakoui et al., J. Virol. (1993) 67:1385-1395; Tomei et al., J. Virol. (1993) 67:4017-4026) and clipping and proteolysis may occur at the N- and C-termini of the E1 and E2 polypeptides. Thus, an E2 polypeptide for use herein may comprise at least amino acids 405-661, e.g., 400, 401, 402 . . . to 661, such as 383 or 384-661, 383 or 384-715, 383 or 384-746, 383 or 384-749 or 383 or 384-809, or 383 or 384 to any C-terminus between 661-809, of an HCV polyprotein, numbered relative to the full-length HCV-1 polyprotein. Similarly, preferable E1 polypeptides for use herein can comprise amino acids 192-326, 192-330, 192-333, 192-360, 192-363, 192-383, or 192 to any C-terminus between 326-383, of an HCV polyprotein.

The E1 E2 complexes may also be made up of immunogenic fragments of E1 and E2 which comprise epitopes. For example, fragments of E1 polypeptides can comprise from about 5 to nearly the full-length of the molecule, such as 6, 10, 25, 50, 75, 100, 125, 150, 175, 185 or more amino acids of an E1 polypeptide, or any integer between the stated numbers. Similarly, fragments of E2 polypeptides can comprise 6, 10, 25, 50, 75, 100, 150, 200, 250, 300, or 350 amino acids of an E2 polypeptide, or any integer between the stated numbers. The E1 and E2 polypeptides may be from the same or different HCV strains.

For example, epitopes derived from, e.g., the hypervariable region of E2, such as a region spanning amino acids 384-410 or 390-410, can be included in the E2 polypeptide. A particularly effective E2 epitope to incorporate into the E2 sequence is one which includes a consensus sequence derived from this region, such as the consensus sequence Gly-Ser-Ala-Ala-Arg-Thr-Thr-Ser-Gly-Phe-Val-Ser-Leu-Phe-Ala-Pro-Gly-Ala-Lys-Gln-Asn, which represents a consensus sequence for amino acids 390-410 of the HCV type 1 genome. Additional epitopes of E1 and E2 are known and described in, e.g., Chien et al., International Publication No. WO 93/00365.

Moreover, the E1 and E2 polypeptides of the complex may lack all or a portion of the membrane spanning domain. The membrane anchor sequence functions to associate the polypeptide to the endoplasmic reticulum. Normally, such polypeptides are capable of secretion into growth medium in which an organism expressing the protein is cultured. However, as described in International Publication No. WO 98/50556, such polypeptides may also be recovered intracellularly. Secretion into growth medium is readily determined using a number of detection techniques, including, e.g., polyacrylamide gel electrophoresis and the like, and immunological techniques such as immunoprecipitation assays as described in, e.g., International Publication No. WO 96/04301, published Feb. 15, 1996. With E1, generally polypeptides terminating with about amino acid position 370 and higher (based on the numbering of HCV-1 E1) will be retained by the ER and hence not secreted into growth media. With E2, polypeptides terminating with about amino acid position 731 and higher (also based on the numbering of the HCV-1 E2 sequence) will be retained by the ER and not secreted. (See, e.g., International Publication No. WO 96/04301, published Feb. 15, 1996). It should be noted that these amino acid positions are not absolute and may vary to some degree. Thus, the present invention contemplates the use of E1 and E2 polypeptides which retain the transmembrane binding domain, as well as polypeptides which lack all or a portion of the transmembrane binding domain, including E1 polypeptides terminating at about amino acids 369 and lower, and E2 polypeptides, terminating at about amino acids 730 and lower, are intended to be captured by the present invention. Furthermore, the C-terminal truncation can extend beyond the transmembrane spanning domain towards the N-terminus. Thus, for example, E1 truncations occurring at positions lower than, e.g., 360 and E2 truncations occurring at positions lower than, e.g., 715, are also encompassed by the present invention. All that is necessary is that the truncated E1 and E2 polypeptides remain functional for their intended purpose. However, particularly preferred truncated E1 constructs are those that do not extend beyond about amino acid 300. Most preferred are those terminating at position 360. Preferred truncated E2 constructs are those with C-terminal truncations that do not extend beyond about amino acid position 715. Particularly preferred E2 truncations are those molecules truncated after any of amino acids 715-730, such as 725. If truncated molecules are used, it is preferable to use E1 and E2 molecules that are both truncated.

The E1 and E2 polypeptides and complexes thereof may also be present as asialoglycoproteins. Such asialoglycoproteins are produced by methods known in the art, such as by using cells in which terminal glycosylation is blocked. When these proteins are expressed in such cells and isolated by GNA lectin affinity chromatography, the E1 and E2 proteins aggregate spontaneously. Detailed methods for producing these E1E2 aggregates are described in, e.g., U.S. Pat. No. 6,074,852, incorporated herein by reference in its entirety.

Moreover, the E1E2 complexes may be present as a heterogeneous mixture of molecules, due to clipping and proteolytic cleavage, as described above. Thus, a composition including E1E2 complexes may include multiple species of E1E2, such as E1E2 terminating at amino acid 746 (E1E2₇₄₆), E1E28 terminating at amino acid 809 (E1E2₈₀₉), or any of the other various E1 and E2 molecules described above, such as E2 molecules with N-terminal truncations of from 1-20 amino acids, such as E2 species beginning at amino acid 387, amino acid 402, amino acid 403, etc.

E1 E2 complexes are readily produced recombinantly, either as fusion proteins or by e.g., co-transfecting host cells with constructs encoding for the E1 and E2 polypeptides of interest. Co-transfection can be accomplished either in trans or cis, i.e., by using separate vectors or by using a single vector which bears both of the E1 and E2 genes. If done using a single vector, both genes can be driven by a single set of control elements or, alternatively, the genes can be present on the vector in individual expression cassettes, driven by individual control elements. Following expression, the E1 and E2 proteins will spontaneously associate. Alternatively, the complexes can be formed by mixing the individual proteins together which have been produced separately, either in purified or semi-purified form, or even by mixing culture media in which host cells expressing the proteins, have been cultured, if the proteins are secreted. Finally, the E1E2 complexes of the present invention may be expressed as a fusion protein wherein the desired portion of E1 is fused to the desired portion of E2.

Methods for producing E1E2 complexes from full-length, truncated E1 and E2 proteins which are secreted into media, as well as intracellularly produced truncated proteins, are known in the art. For example, such complexes may be produced recombinantly, as described in U.S. Pat. No. 6,121,020; Ralston et al., J. Virol. (1993) 67:6753-6761, Grakoui et al., J. Virol. (1993) 67:1385-1395; and Lanford et al., Virology (1993) 197:225-235.

Thus, polynucleotides encoding HCV E1 and E2 polypeptides for use with the present invention can be made using standard techniques of molecular biology. For example, polynucleotide sequences coding for the above-described molecules can be obtained using recombinant methods, such as by screening cDNA and genomic libraries from cells expressing the gene, or by deriving the gene from a vector known to include the same. Furthermore, the desired gene can be isolated directly from viral nucleic acid molecules, using techniques described in the art, such as in Houghton et al., U.S. Pat. No. 5,350,671. The gene of interest can also be produced synthetically, rather than cloned. The molecules can be designed with appropriate codons for the particular sequence. The complete sequence is then assembled from overlapping oligonucleotides prepared by standard methods and assembled into a complete coding sequence. See, e.g., Edge (1981) Nature 292:756; Nambair et al. (1984) Science 223:1299; and Jay et al. (1984) J. Biol. Chem. 259:6311.

Thus, particular nucleotide sequences can be obtained from vectors harboring the desired sequences or synthesized completely or in part using various oligonucleotide synthesis techniques known in the art, such as site-directed mutagenesis and polymerase chain reaction (PCR) techniques where appropriate. See, e.g., Sambrook, supra. In particular, one method of obtaining nucleotide sequences encoding the desired sequences is by annealing complementary sets of overlapping synthetic oligonucleotides produced in a conventional, automated polynucleotide synthesizer, followed by ligation with an appropriate DNA ligase and amplification of the ligated nucleotide sequence via PCR. See, e.g., Jayaraman et al. (1991) Proc. Natl. Acad. Sci. USA 88:4084-4088. Additionally, oligonucleotide directed synthesis (Jones et al. (1986) Nature 54:75-82), oligonucleotide directed mutagenesis of pre-existing nucleotide regions (Riechmann et al. (1988) Nature 332:323-327 and Verhoeyen et al. (1988) Science 239:1534-1536), and enzymatic filling-in of gapped oligonucleotides using T₄ DNA polymerase (Queen et al. (1989) Proc. Natl. Acad. Sci. USA 86:10029-10033) can be used to provide molecules having altered or enhanced antigen-binding capabilities and immunogenicity.

Once coding sequences have been prepared or isolated, such sequences can be cloned into any suitable vector or replicon. Numerous cloning vectors are known to those of skill in the art, and the selection of an appropriate cloning vector is a matter of choice. Suitable vectors include, but are not limited to, plasmids, phages, transposons, cosmids, chromosomes or viruses which are capable of replication when associated with the proper control elements.

The coding sequence is then placed under the control of suitable control elements, depending on the system to be used for expression. Thus, the coding sequence can be placed under the control of a promoter, ribosome binding site (for bacterial expression) and, optionally, an operator, so that the DNA sequence of interest is transcribed into RNA by a suitable transformant. The coding sequence may or may not contain a signal peptide or leader sequence which can later be removed by the host in post-translational processing. See, e.g., U.S. Pat. Nos. 4,431,739; 4,425,437; 4,338,397.

In addition to control sequences, it may be desirable to add regulatory sequences which allow for regulation of the expression of the sequences relative to the growth of the host cell. Regulatory sequences are known to those of skill in the art, and examples include those which cause the expression of a gene to be turned on or off in response to a chemical or physical stimulus, including the presence of a regulatory compound. Other types of regulatory elements may also be present in the vector. For example, enhancer elements may be used herein to increase expression levels of the constructs. Examples include the SV40 early gene enhancer (Dijkema et al. (1985) EMBO J. 4:761), the enhancer/promoter derived from the long terminal repeat (LTR) of the Rous Sarcoma Virus (Gorman et al. (1982) Proc. Natl. Acad. Sci. USA 79:6777) and elements derived from human CMV (Boshart et al. (1985) Cell 41:521), such as elements included in the CMV intron A sequence (U.S. Pat. No. 5,688,688). The expression cassette may further include an origin of replication for autonomous replication in a suitable host cell, one or more selectable markers, one or more restriction sites, a potential for high copy number and a strong promoter.

An expression vector is constructed so that the particular coding sequence is located in the vector with the appropriate regulatory sequences, the positioning and orientation of the coding sequence with respect to the control sequences being such that the coding sequence is transcribed under the “control” of the control sequences (i.e., RNA polymerase which binds to the DNA molecule at the control sequences transcribes the coding sequence). Modification of the sequences encoding the molecule of interest may be desirable to achieve this end. For example, in some cases it may be necessary to modify the sequence so that it can be attached to the control sequences in the appropriate orientation; i.e., to maintain the reading frame. The control sequences and other regulatory sequences may be ligated to the coding sequence prior to insertion into a vector. Alternatively, the coding sequence can be cloned directly into an expression vector which already contains the control sequences and an appropriate restriction site.

As explained above, it may also be desirable to produce mutants or analogs of the polypeptide of interest. Mutants or analogs of HCV polypeptides for use in the subject compositions may be prepared by the deletion of a portion of the sequence encoding the polypeptide of interest, by insertion of a sequence, and/or by substitution of one or more nucleotides within the sequence. Techniques for modifying nucleotide sequences, such as site-directed mutagenesis, and the like, are well known to those skilled in the art. See, e.g., Sambrook et al., supra; Kunkel, T. A. (1985) Proc. Natl. Acad. Sci. USA (1985) 82:448; Geisselsoder et al. (1987) BioTechniques 5:786; Zoller and Smith (1983) Methods Enzymol. 100:468; Dalbie-McFarland et al. (1982) Proc. Natl. Acad. Sci USA 79:6409.

The molecules can be expressed in a wide variety of systems, including insect, mammalian, bacterial, viral and yeast expression systems, all well known in the art.

For example, insect cell expression systems, such as baculovirus systems, are known to those of skill in the art and described in, e.g., Summers and Smith, Texas Agricultural Experiment Station Bulletin No. 1555 (1987). Materials and methods for baculovirus/insect cell expression systems are commercially available in kit form from, inter alia, Invitrogen, San Diego Calif. (“MaxBac” kit). Similarly, bacterial and mammalian cell expression systems are well known in the art and described in, e.g., Sambrook et al., supra. Yeast expression systems are also known in the art and described in, e.g., Yeast Genetic Engineering (Barr et al., eds., 1989) Butterworths, London.

A number of appropriate host cells for use with the above systems are also known. For example, mammalian cell lines are known in the art and include immortalized cell lines available from the American Type Culture Collection (ATCC), such as, but not limited to, Chinese hamster ovary (CHO) cells, HeLa cells, baby hamster kidney (BHK) cells, monkey kidney cells (COS), human embryonic kidney cells, human hepatocellular carcinoma cells (e.g., Hep G2), Madin-Darby bovine kidney (“MDBK”) cells, as well as others. Similarly, bacterial hosts such as E. coli, Bacillus subtilis, and Streptococcus spp., will find use with the present expression constructs. Yeast hosts useful in the present invention include inter alia, Saccharomyces cerevisiae, Candida albicans, Candida maltosa, Hansenula polymorpha, Kluyveromyces fragilis, Kluyveromyces lactis, Pichia guillerimondii, Pichia pastoris, Schizosaccharomyces pombe and Yarrowia lipolytica. Insect cells for use with baculovirus expression vectors include, inter alia, Aedes aegypti, Autographa californica, Bombyx mori, Drosophila melanogaster, Spodoptera frugiperda, and Trichoplusia ni.

Nucleic acid molecules comprising nucleotide sequences of interest can be stably integrated into a host cell genome or maintained on a stable episomal element in a suitable host cell using various gene delivery techniques well known in the art. See, e.g., U.S. Pat. No. 5,399,346.

Depending on the expression system and host selected, the molecules are produced by growing host cells transformed by an expression vector described above under conditions whereby the protein is expressed. The expressed protein is then isolated from the host cells and purified. If the expression system secretes the protein into growth media, the product can be purified directly from the media. If it is not secreted, it can be isolated from cell lysates. The selection of the appropriate growth conditions and recovery methods are within the skill of the art.

Compositions

Once produced, the E1E2 antigens may be provided in vaccine compositions, in e.g., prophylactic (i.e., to prevent infection) or therapeutic (to treat HCV following infection) vaccines. The vaccines can comprise mixtures of one or more of the E1E2 complexes, such as E1E2 complexes derived from more than one viral isolate, as well as additional HCV antigens. Moreover, as explained above, the E1E2 complexes may be present as a heterogeneous mixture of molecules, due to clipping and proteolytic cleavage. Thus, a composition including E1E2 complexes may include multiple species of E1E2, such as E1E2 terminating at amino acid 746 (E1E2₇₄₆), E1E28 terminating at amino acid 809 (E1E2₈₀₉), or any of the other various E1 and E2 molecules described above, such as E2 molecules with N-terminal truncations of from 1-20 amino acids, such as E2 species beginning at amino acid 387, amino acid 402, amino acid 403, etc.

The vaccines may be administered in conjunction with other antigens and immunoregulatory agents, for example, immunoglobulins, cytokines, lymphokines, and chemokines, including but not limited to cytokines such as IL-2, modified IL-2 (cys125-ser125), GM-CSF, IL-12, γ-interferon, IP-10, MIP1β, FLP-3, ribavirin and RANTES.

The vaccines will generally include one or more “pharmaceutically acceptable excipients or vehicles” such as water, saline, glycerol, ethanol, etc. Additionally, auxiliary substances, such as wetting or emulsifying agents, pH buffering substances, and the like, may be present in such vehicles.

A carrier is optionally present which is a molecule that does not itself induce the production of antibodies harmful to the individual receiving the composition. Suitable carriers are typically large, slowly metabolized macromolecules such as proteins, polysaccharides, polylactic acids, polyglycollic acids, polymeric amino acids, amino acid copolymers, lipid aggregates (such as oil droplets or liposomes), and inactive virus particles. Such carriers are well known to those of ordinary skill in the art. Furthermore, the HCV polypeptide may be conjugated to a bacterial toxoid, such as toxoid from diphtheria, tetanus, cholera, etc.

As explained herein, submicron oil-in-water emulsions and/or ISSs, such as CpG oligonucleotides (described further below), may be present in the same composition to enhance the immune response. Additional adjuvants may also be present, such as but are not limited to: (1) aluminum salts (alum), such as aluminum hydroxide, aluminum phosphate, aluminum sulfate, etc.; (2) Ribi™ adjuvant system (RAS), (Ribi Immunochem, Hamilton, Mont.) containing 2% Squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox™); (3) saponin adjuvants, such as QS21 or Stimulon™ (Cambridge Bioscience, Worcester, Mass.) may be used or particles generated therefrom such as ISCOMs (immunostimulating complexes), which ISCOMs may be devoid of additional detergent (see, e.g., International Publication No. WO 00/07621); (4) Complete Freunds Adjuvant (CFA) and Incomplete Freunds Adjuvant (IFA); (5) cytokines, such as interleukins, such as IL-1, IL-2, IL-4, IL-5, IL-6, IL-7, IL-12 etc. (see, e.g., International Publication No. WO 99/44636), interferons, such as gamma interferon, macrophage colony stimulating factor (M-CSF), tumor necrosis factor (TNF), etc.; (6) detoxified mutants of a bacterial ADP-ribosylating toxin such as a cholera toxin (CT), a pertussis toxin (PT), or an E. coli heat-labile toxin (LT), particularly LT-K63 (where lysine is substituted for the wild-type amino acid at position 63) LT-R72 (where arginine is substituted for the wild-type amino acid at position 72), CT-S 109 (where serine is substituted for the wild-type amino acid at position 109), and PT-K9/G129 (where lysine is substituted for the wild-type amino acid at position 9 and glycine substituted at position 129) (see, e.g., International Publication Nos. WO93/13202 and WO92/19265); (7) monophosporyl lipid A (MPL) or 3-O-deacylated MPL (3dMPL) (see, e.g., GB 2220221; EPA 0689454), optionally in the substantial absence of alum (see, e.g., International Publication No. WO 00/56358); (8) combinations of 3dMPL with, for example, QS21 and/or oil-in-water emulations (see, e.g., EPA 0835318; EPA 0735898; EPA 0761231); (9) a polyoxyethylene ether or a polyoxyethylene ester (see, e.g., International Publication No. WO 99/52549); (10) a saponin and an immunostimulatory oligonucleotide, such as a CpG oligonucleotide (see, e.g., International Publication No. WO 00/62800); (11) an immunostimulant and a particle of a metal salt (see, e.g., International Publication No. WO 00/23105); (12) a saponin and an oil-in-water emulsion (see, e.g., International Publication No. WO 99/11241; (13) a saponin (e.g., QS21)+3dMPL+IL-12 (optionally+a sterol) (see, e.g., International Publication No. WO 98/57659); and (14) other substances that act as immunostimulating agents to enhance the effectiveness of the composition.

Muramyl peptides include, but are not limited to, N-acetyl-muramyl-L-threonyl-D-isoglutamine (thr-MDP), N-acteyl-normuramyl-L-alanyl-D-isogluatme (nor-MDP), -acetylmuramyl-L-alanyl-D-isogluatminyl-L-alanine-2-(1′-2′-dipalmitoyl-sn-glycero-3-huydroxyphosphoryloxy)-ethylamine (MTP-PE), etc.

Typically, the vaccine compositions are prepared as injectables, either as liquid solutions or suspensions; solid forms suitable for solution in, or suspension in, liquid vehicles prior to injection may also be prepared.

The vaccines will comprise a therapeutically effective amount of the E1E2 complexes and any other of the above-mentioned components, as needed. By “therapeutically effective amount” is meant an amount of an E1E2 protein which will induce an immunological response, preferably a protective immunological response, in the individual to which it is administered. Such a response will generally result in the development in the subject of a secretory, cellular and/or antibody-mediated immune response to the vaccine. Usually, such a response includes but is not limited to one or more of the following effects; the production of antibodies from any of the immunological classes, such as immunoglobulins A, D, E, G or M; the proliferation of B and T lymphocytes; the provision of activation, growth and differentiation signals to immunological cells; expansion of helper T cell, suppressor T cell, and/or cytotoxic T cell and/or γδ T cell populations.

Once formulated, the vaccines are conventionally administered parenterally, e.g., by injection, either subcutaneously or intramuscularly. Additional formulations suitable for other modes of administration include oral and pulmonary formulations, suppositories, and transdermal applications. Dosage treatment may be a single dose schedule or a multiple dose schedule. Preferably, the effective amount is sufficient to bring about treatment or prevention of disease symptoms. The exact amount necessary will vary depending on the subject being treated; the age and general condition of the individual to be treated; the capacity of the individual's immune system to synthesize antibodies; the degree of protection desired; the severity of the condition being treated; the particular E1E2 polypeptide selected and its mode of administration, among other factors. An appropriate effective amount can be readily determined by one of skill in the art. A “therapeutically effective amount” will fall in a relatively broad range that can be determined through routine trials using in vitro and in vivo models known in the art. The amount of E1E2 polypeptides used in the examples below provides general guidance which can be used to optimize the elicitation of anti-E1, anti-E2 and/or anti-E1E2 antibodies.

In particular, an E1E2 complex is preferably injected intramuscularly to a large mammal, such as a primate, for example, a baboon, chimpanzee, or human, at a dose of approximately 0.1 μg to about 5.0 mg per dose, or any amount between the stated ranges, such as 0.5 μg to about 1.0 μg, 1 μg to about 500 μg, 2.5 μg to about 250 μg, 4 μg to about 200 μg, such as 4, 5, 6, 7, 8, 9, 10 . . . 20 . . . 30 . . . 40 . . . 50 . . . 60 . . . 70 . . . 80 . . . 90 . . . 100, etc., μg per dose. E1E2 polypeptides can be administered either to a mammal that is not infected with an HCV or can be administered to an HCV-infected mammal.

Administration of E1E2 polypeptides can elicit an anti-E1, anti-E2 and/or anti-E1E2 antibody titer in the mammal that lasts for at least 1 week, 2 weeks, 1 month, 2 months, 3 months, 4 months, 6 months, 1 year, or longer. E1E2 polypeptides can also be administered to provide a memory response. If such a response is achieved, antibody titers may decline over time, however exposure to the HCV virus or immunogen results in the rapid induction of antibodies, e.g., within only a few days. Optionally, antibody titers can be maintained in a mammal by providing one or more booster injections of the E1E2 polypeptides at 2 weeks, 1 month, 2 months, 3 months, 4 months, 5 months, 6 months, 1 year, or more after the primary injection.

Preferably, an E1E2 polypeptide elicits an antibody titer of at least 10, 100, 150, 175, 200, 300, 400, 500, 750, 1,000, 1,500, 2,000, 3,000, 5,000, 10,000, 20,000, 30,000, 40,000, 50,000 (geometric mean titer), or higher, or any number between the stated titer, as determined using a standard immunoassay, such as the immunoassay described in the examples below. See, e.g., Chien et al., Lancet (1993) 342:933; and Chien et al., Proc. Natl. Acad. Sci. USA (1992) 89:10011.

Submicron Oil-in-Water Emulsions

As explained above, a submicron oil-in-water emulsion formulation may also be administered to the vertebrate subject, either prior to, concurrent with, or subsequent to, delivery of the E1E2 antigen. Submicron oil-in water emulsions for use herein include nontoxic, metabolizable oils and commercial emulsifiers. Examples of nontoxic, metabolizable oils include, without limitation, vegetable oils, fish oils, animal oils or synthetically prepared oils. Fish oils, such as cod liver oil, shark liver oils and whale oils, are preferred, with squalene, 2,6,10,15,19,23-hexamethyl-2,6,10,14,18,22-tetracosahexaene, found in shark liver oil, particularly preferred. The oil component will be present in an amount of from about 0.5% to about 20% by volume, preferably in an amount up to about 15%, more preferably in an amount of from about 1% to about 12% and most preferably from 1% to about 4% oil.

The aqueous portion of the adjuvant can be buffered saline or unadulterated water. Since the compositions are intended for parenteral administration, it is preferable to make up the final solutions so that the tonicity, i.e., osmolality, is essentially the same as normal physiological fluids, in order to prevent post-administration swelling or rapid absorption of the composition due to differential ion concentrations between the composition and physiological fluids. If saline is used rather than water, it is preferable to buffer the saline in order to maintain a pH compatible with normal physiological conditions. Also, in certain instances, it may be necessary to maintain the pH at a particular level in order to insure the stability of certain composition components. Thus, the pH of the compositions will generally be pH 6-8 and pH can be maintained using any physiologically acceptable buffer, such as phosphate, acetate, tris, bicarbonate or carbonate buffers, or the like. The quantity of the aqueous agent present will generally be the amount necessary to bring the composition to the desired final volume.

Emulsifying agents suitable for use in the oil-in-water formulations include, without limitation, sorbitan-based non-ionic surfactants such as a sorbitan mono-, di-, or triester, for example those commercially available under the name of Span™ or Arlacel™, such as Span™ 85 (sorbitan trioleate); polyoxyethylene sorbitan mono-, di-, or triesters commercially known by the name Tween™, such as Tween 80™ (polyoxyelthylenesorbitan monooleate); polyoxyethylene fatty acids available under the name Myrj™; polyoxyethylene fatty acid ethers derived from lauryl, acetyl, stearyl and oleyl alcohols, such as those known by the name of Brij™; and the like. These substances are readily available from a number of commercial sources, including Sigma, St. Louis, Mo. and ICI America's Inc., Wilmington, Del. These emulsifying agents may be used alone or in combination. The emulsifying agent will usually be present in an amount of 0.02% to about 2.5% by weight (w/v), preferably 0.05% to about 1%, and most preferably 0.01% to about 0.5. The amount present will generally be about 20-30% of the weight of the oil used.

The emulsions can also contain other immunostimulating agents, such as muramyl peptides, including, but not limited to, N-acetyl-muramyl-L-threonyl-D-isoglutamine (thr-MDP), N-acteyl-normuramyl-L-alanyl-D-isogluatme (nor-MDP), -acetylmuramyl-L-alanyl-D-isogluatminyl-L-alanine-2-(1′-2′-dipalmitoyl-sn-glycero-3-huydroxyphosphoryloxy)-ethylamine (MTP-PE), etc. Immunostimulating bacterial cell wall components, such as monophosphorylipid A (MPL), trehalose dimycolate (TDM), and cell wall skeleton (CWS), may also be present. Alternatively, the emulsions may be free of these agents, such as free of MTP-PE. The submicron oil-in-water emulsions of the present invention may also be devoid of any polyoxypropylene-polyoxyethylene (POP-POE) block copolymers. For a description of various suitable submicron oil-in-water emulsion formulations for use with the present invention, as well as immunostimulating agents, see, e.g., International Publication No. WO 90/14837; Remington: The Science and Practice of Pharmacy, Mack Publishing Company, Easton, Pa., 19th edition, 1995; Van Nest et al., “Advanced adjuvant formulations for use with recombinant subunit vaccines,” In Vaccines 92, Modern Approaches to New Vaccines (Brown et al., ed.) Cold Spring Harbor Laboratory Press, pp. 57-62 (1992); Ott et al., “MF59—Design and Evaluation of a Safe and Potent Adjuvant for Human Vaccines” in Vaccine Design: The Subunit and Adjuvant Approach (Powell, M. F. and Newman, M. J. eds.) Plenum Press, New York (1995) pp. 277-296; and U.S. Pat. No. 6,299,884, incorporated herein by reference in its entirety.

In order to produce submicron particles, i.e., particles less than 1 micron in diameter and in the nanometer size range, a number of techniques can be used. For example, commercial emulsifiers can be used that operate by the principle of high shear forces developed by forcing fluids through small apertures under high pressure. Examples of commercial emulsifiers include, without limitation, Model 110Y microfluidizer (Microfluidics, Newton, Mass.), Gaulin Model 30CD (Gaulin, Inc., Everett, Mass.), and Rainnie Minilab Type 8.30H (Miro Atomizer Food and Dairy, Inc., Hudson, Wis.). The appropriate pressure for use with an individual emulsifier is readily determined by one of skill in the art. For example, when the Model 110Y microfluidizer is used, operation at 5000 to 30,000 psi produces oil droplets with diameters of about 100 to 750 nm.

The size of the oil droplets can be varied by changing the ratio of detergent to oil (increasing the ratio decreases droplet size), operating pressure (increasing operating pressure reduces droplet size), temperature (increasing temperature decreases droplet size), and adding an amphipathic immunostimulating agent (adding such agents decreases droplet size). Actual droplet size will vary with the particular detergent, oil and immunostimulating agent (if any) and with the particular operating conditions selected. Droplet size can be verified by use of sizing instruments, such as the commercial Sub-Micron Particle Analyzer (Model N4MD) manufactured by the Coulter Corporation, and the parameters can be varied using the guidelines set forth above until substantially all droplets are less than 1 micron in diameter, preferably less than about 0.8 microns in diameter, and most preferably less than about 0.5 microns in diameter. By substantially all is meant at least about 80% (by number), preferably at least about 90%, more preferably at least about 95%, and most preferably at least about 98%. The particle size distribution is typically Gaussian, so that the average diameter is smaller than the stated limits.

Particularly preferred submicron oil-in-water emulsions for use herein are squalene/water emulsions optionally containing varying amounts of MTP-PE, such as a submicron oil-in-water emulsions containing 4-5% w/v squalene, 0.25-1.0% w/v Tween 80™ (polyoxyelthylenesorbitan monooleate), and/or 0.25-1.0% Span 85™ (sorbitan trioleate), and optionally, N-acetylmuramyl-L-alanyl-D-isogluatminyl-L-alanine-2-(1′-2′-dipalmitoyl-sn-glycero-3-huydroxyphosphoryloxy)-ethylamine (MTP-PE), for example, the submicron oil-in-water emulsion known as “MF59” (International Publication No. WO 90/14837; U.S. Pat. No. 6,299,884, incorporated herein by reference in its entirety; and Ott et al., “MF59—Design and Evaluation of a Safe and Potent Adjuvant for Human Vaccines” in Vaccine Design: The Subunit and Adjuvant Approach (Powell, M. F. and Newman, M. J. eds.) Plenum Press, New York, 1995, pp. 277-296). MF59 contains 4-5% w/v Squalene (e.g., 4.3%), 0.25-0.5% w/v Tween 80™, and 0.5% w/v Span 85™ and optionally contains various amounts of MTP-PE, formulated into submicron particles using a microfluidizer such as Model 110Y microfluidizer (Microfluidics, Newton, Mass.). For example, MTP-PE may be present in an amount of about 0-500 μg/dose, more preferably 0-250 μg/dose and most preferably, 0-100 μg/dose. As used herein, the term “MF59-0” refers to the above submicron oil-in-water emulsion lacking MTP-PE, while the term MF59-MTP denotes a formulation that contains MTP-PE. For instance, “MF59-100” contains 100 μg MTP-PE per dose, and so on. MF69, another submicron oil-in-water emulsion for use herein, contains 4.3% w/v squalene, 0.25% w/v Tween 80™, and 0.75% w/v Span 85™ and optionally MTP-PE. Yet another submicron oil-in-water emulsion is MF75, also known as SAF, containing 10% squalene, 0.4% Tween 80™, 5% pluronic-blocked polymer L121, and thr-MDP, also microfluidized into a submicron emulsion. MF75-MTP denotes an MF75 formulation that includes MTP, such as from 100-400 μg MTP-PE per dose.

Submicron oil-in-water emulsions, methods of making the same and immunostimulating agents, such as muramyl peptides, for use in the compositions, are described in detail in International Publication No. WO 90/14837 and commonly owned, allowed, U.S. patent application Ser. No. 08/418,870, incorporated herein by reference in its entirety.

Once the submicron oil-in-water emulsion is formulated it can be administered to the vertebrate subject, either prior to, concurrent with, or subsequent to, delivery of the antigen, and the ISS, if used. If administered prior to immunization with the antigen, the adjuvant formulations can be administered as early as 5-10 days prior to immunization, preferably 3-5 days prior to immunization and most preferably 1-3 or 2 days prior to immunization with the antigens of interest. If administered separately, the submicron oil-in-water formulation can be delivered either to the same site of delivery as the antigen compositions or to a different delivery site.

If simultaneous delivery is desired, the submicron oil-in-water formulation can be included with the antigen compositions. Generally, the antigens and submicron oil-in-water emulsion can be combined by simple mixing, stirring, or shaking. Other techniques, such as passing a mixture of the two components rapidly through a small opening (such as a hypodermic needle) can also be used to provide the vaccine compositions.

If combined, the various components of the composition can be present in a wide range of ratios. For example, the antigen and emulsion components are typically used in a volume ratio of 1:50 to 50:1, preferably 1:10 to 10:1, more preferably from about 1:5 to 3:1, and most preferably about 1:1. However, other ratios may be more appropriate for specific purposes, such as when a particular antigen has a low immungenicity, in which case a higher relative amount of the antigen component is required.

Immunostimulatory Nucleic Acid Molecules (ISS)

Bacterial DNA has previously been reported to stimulate mammalian immune responses. See, e.g., Krieg et al., Nature (1995) 374:546-549. This immunostimulatory ability has been attributed to the high frequency of immunostimulatory nucleic acid molecules (ISSs), such as unmethylated CpG dinucleotides present in bacterial DNA. Oligonucleotides containing unmethylated CpG motifs have been shown to induce activation of B cells, NK cells and antigen-presenting cells (APCs), such as monocytes and macrophages. See, e.g., U.S. Pat. No. 6,207,646.

The present invention makes use of adjuvants derived from ISSs. The ISS of the invention includes an oligonucleotide which can be part of a larger nucleotide construct such as plasmid or bacterial DNA. The oligonucleotide can be linearly or circularly configured, or can contain both linear and circular segments. The oligonucleotide may include modifications such as, but are not limited to, modifications of the YOH or 5′OH group, modifications of the nucleotide base, modifications of the sugar component, and modifications of the phosphate group. The ISS can comprise ribonucleotides (containing ribose as the only or principal sugar component), deoxyribonucleotides (containing deoxyribose as the principal sugar component). Modified sugars or sugar analogs may also be incorporated in the oligonucleotide. Examples of sugar moieties that can be used include ribose, deoxyribose, pentose, deoxypentose, hexose, deoxyhexose, glucose, arabinose, xylose, lyxose, and a sugar analog cyclopentyl group. The sugar may be in pyranosyl or in a furanosyl form. A phosphorous derivative (or modified phosphate group) can be used and can be a monophosphate, diphosphate, triphosphate, alkylphosphate, alkanephosphate, phosphoronthioate, phosphorodithioate, or the like. Nucleic acid bases that are incorporated in the oligonucleotide base of the ISS can be naturally occurring purine and pyrimidine bases, namely, uracil or thymine, cytosine, adenine and guanine, as well as naturally occurring and synthetic modifications of these bases. Moreover, a large number of non-natural nucleosides comprising various heterocyclic bases and various sugar moieties (and sugar analogs) are available, and known to those of skill in the art.

Structurally, the root oligonucleotide of the ISS is a CG-containing nucleotide sequence or a p(1C) nucleotide sequence, which may be palindromic. The cytosine may be methylated or unmethylated. Examples of particular ISS molecules for use in the present invention include CpG, CpY and CpR molecules, and the like, known in the art.

Preferred ISSs are those derived from the CpG family of molecules, CpG dinucleotides and synthetic oligonucleotides which comprise CpG motifs (see, e.g., Krieg et al. Nature (1995) 374:546 and Davis et al. J. Immunol. (1998) 160:870-876), such as any of the various immunostimulatory CpG oligonucleotides disclosed in U.S. Pat. No. 6,207,646, incorporated herein by reference in its entirety. Such CpG oligonucleotides generally comprise at least 8 up to about 100 nucleotides, preferably 8 to 40 nucleotides, more preferably 15-35 nucleotides, preferably 15-25 nucleotides, and any number of nucleotides between these values. For example, oligonucleotides comprising the consensus CpG motif, represented by the formula 5′-X₁CGX₂-3′, where X₁ and X₂ are nucleotides and C is unmethylated, will find use as immunostimulatory CpG molecules. Generally, X₁ is A, G or T, and X₂ is C or T. Other useful CpG molecules include those captured by the formula 5′-X₁X₂CGX₃X₄, where X, and X₂ are a sequence such as GpT, GpG, GpA, ApA, ApT, ApG, CpT, CpA, CpG, TpA, TpT or TpG, and X₃ and X₄ are TpT, CpT, ApT, ApG, CpG, TpC, ApC, CpC, TpA, ApA, GpT, CpA, or TpG, wherein “p” signifies a phosphate bond. Preferably, the oligonucleotides do not include a GCG sequence at or near the 5′- and/or 3′ terminus. Additionally, the CpG is preferably flanked on its 5′-end with two purines (preferably a GpA dinucleotide) or with a purine and a pyrimidine (preferably, GpT), and flanked on its 3′-end with two pyrimidines, preferably a TpT or TpC dinucleotide. Thus, preferred molecules will comprise the sequence GACGTT, GACGTC, GTCGTT or GTCGCT, and these sequences will be flanked by several additional nucleotides, such as with 1-20 or more nucleotides, preferably 2 to 10 nucleotides and more preferably, 3 to 5 nucleotides, or any integer between these stated ranges. The nucleotides outside of the central core area appear to be extremely amendable to change.

Moreover, the CpG oligonucleotides for use herein may be double- or single-stranded. Double-stranded molecules are more stable in vivo while single-stranded molecules display enhanced immune activity. Additionally, the phosphate backbone may be modified, such as phosphorodithioate-modified, in order to enhance the immunostimulatory activity of the CpG molecule. As described in U.S. Pat. No. 6,207,646, CpG molecules with phosphorothioate backbones preferentially activate B-cells, while those having phosphodiester backbones preferentially activate monocytic (macrophages, dendritic cells and monocytes) and NK cells.

Exemplary CpG oligonucleotides for use in the present compositions include molecules with the sequence 5′-TCCATGACGTTCCTGACGTT-3′ (SEQ ID NO:1) and 5′-TCGTCGTTTTGTCGTTTTGTCGTT-3′ (SEQ ID NO:5).

ISS molecules can readily be tested for their ability to stimulate an immune response using standard techniques, well known in the art. For example, the ability of the molecule to stimulate a humoral and/or cellular immune response is readily determined using the immunoassays described above. Moreover, the antigen and submicron oil-in-water compositions can be administered with and without the ISSs to determine whether an immune response is enhanced.

As explained above, the ISS can be administered either prior to, concurrent with, or subsequent to, delivery of the antigen and/or the submicron oil-in-water emulsion. If administered prior to immunization with the antigen and/or the submicron oil-in-water emulsion, the ISS can be administered as early as 5-10 days prior to immunization, preferably 3-5 days prior to immunization and most preferably 1-3 or 2 days prior to immunization. If administered separately, the ISS can be delivered either to the same site of delivery as the antigen compositions or to a different delivery site. If simultaneous delivery is desired, the ISS can be included with the antigen compositions.

Generally about 0.5 μg to 5000 μg of the ISS will be used, more generally 0.5 μg to about 1000, preferably 0.5 μg to about 500 μg, or from 1 to about 100 μg, preferably about 5 to about 50 μg, preferably 5 to about 30, or any amount within these ranges, of the ISS per dose, will find use with the present methods.

Deposits of Strains Useful in Practicing the Invention

A deposit of biologically pure cultures of the following strains was made with the American Type Culture Collection, 10801 University Boulevard, Manassas, Va., under the provisions of the Budapest Treaty. The accession number indicated was assigned after successful viability testing, and the requisite fees were paid. The designated deposits will be maintained for a period of thirty (30) years from the date of deposit, or for five (5) years after the last request for the deposit, whichever is longer. Should a culture become nonviable or be inadvertently destroyed, or, in the case of plasmid-containing strains, lose its plasmid, it will be replaced with a viable culture(s) of the same taxonomic description.

These deposits are provided merely as convenience to those of skill in the art, and are not an admission that a deposit is required under 35 USC §112. Should there be a discrepancy between the sequence presented in the present application and the sequence of the gene of interest in the deposited plasmid due to routine sequencing errors, the sequence in the deposited plasmid controls. A license may be required to make, use, or sell the deposited materials, and no such license is hereby granted.

| Strain | Deposit Date | ATCC No. |
|---|---|---|
| E1E2₈₀₉ in CHO cells | Aug. 16, 2001 | PTA-3643 |

III. Experimental

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### EXAMPLE 1

### Production of HCV E1E2

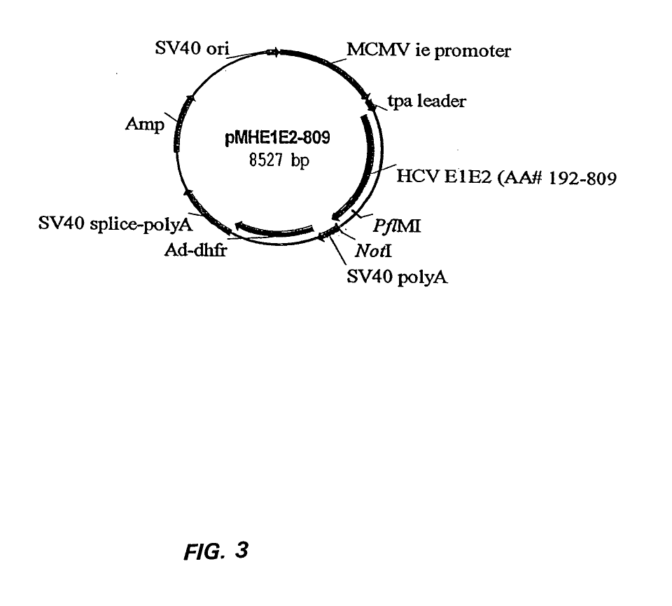

An HCV E1E2 complex for use in the present vaccine compositions was prepared as a fusion protein as follows. In particular, mammalian expression plasmid pMH-E1E2-809 (FIG. 3; ATCC Accession No. PTA 3643) encodes an E1E2 fusion protein which includes amino acids 192-809 of HCV-1 (see, Choo et al., Proc. Natl. Acad. Sci. USA (1991) 88:2451-2455). The sequence of the E1E2₈₀₉ molecule is shown in FIGS. 2A-2C herein.

Chinese Hamster Ovary (CHO) cells were used for expression of the HCV E1E2 sequence from pMH-E1E2-809. In particular, CHO DG44 cells were used. These cells, described by Uraub et al., Proc. Natl. Acad. Sci. USA (1980) 77:4216-4220, were derived from CHO K-1 cells and were made dihydrofolate reductase (dhfr) deficient by virtue of a double deletion in the dhfr gene.

DG44 cells were transfected with pMH-E1E2-809. The transfected cells were grown in selective medium such that only those cells expressing the dhfr gene could grow (Sambrook et al., supra). Isolated CHO colonies were picked (˜800 colonies) into individual wells of a 96-well plate. From the original 96-well plates, replicates were made to perform expression experiments. The replicate plates were grown until the cells made a confluent monolayer. The cells were fixed to the wells of the plate and permeablized using cold methanol. 3D5C3, a monoclonal antibody against E1E2, and 3E5-1 a monoclonal antibody against E2, were used to probe the fixed cells. After adding an anti-mouse HRP conjugate, followed by substrate, the cell lines with the highest expression were determined. The highest expressing cell lines were then expanded to 24-well cluster plates. The assay for expression was repeated, and again, the highest expressing cell lines were expanded to wells of greater volume. This was repeated until the highest expressing cell lines were expanded from 6-well plates into tissue culture flasks. At this point there was sufficient quantity of cells to allow accurate count and harvest of the cells, and quantitative expression assays were done. An ELISA (Spaete et al., Virol (1992) 188:819-830) was performed on the cell extract, to determine high expressors.

### EXAMPLE 2

### Purification of HCV E1E2

Following expression, CHO cells were lysed and the intracellularly produced E1E2₈₀₉ was purified by GNA-lectin affinity chromatography (GNA step), followed by hydroxyapatite (HAP) column chromatography (HAP step), DV50 membrane filtration (DV50 step), SP Sepharose HP column chromatography (SP step), Q membrane filtration (Q step) and G25 Sephadex column chromatography G25 step). At the completion of each of the processing steps, the product pool was either 0.2μ filtered and held at 2-8° C. or processed immediately through the next purification step. At the completion of the purification process, the antigen was 0.2μ filtered and held frozen at −60° C., or lower until filtered for formulation.

Specifically, to lyse the cells, two volumes of chilled lysis buffer (1% Triton X-100 in 100 mM Tris, pH8, and 1 mM EDTA) were added to the CHO cells at 2-8° C. The mixture was centrifuged at 5000 rpm for 45 min at 2-8° C. to remove debris. The supernatant was collected and filtered through a Sartorias 0.65 μm Sartopure prefilter (Sartorius) then a Sartorias 0.65 mm Sartofine prefilter, followed by a Sartorious 0.45 μm Sartobran filter and a 0.2 μm Sartobran filter. The filtered lysate was kept on ice prior to loading on the GNA column.

A GNA agarose column (1885 ml, 200×600, Vector Labs, Burlingame, Calif.) was pre-equilibrated with eight column volumes of equilibration buffer (25 mM NaPO₄, 1.0 M NaCl, 12% Triton X-100, pH 6.8) prior to loading. The lysate was applied to the column at 31.4 ml/min (6 cm/hr) over night. The column was washed with 4 bed volumes of equilibration buffer, then washed again with 5 bed volumes of 10 mM NaPO₄, 80 mM NaCl, 0.1% Triton X-100, pH 6.8. The product was eluted with 1 M methyl α-D-mannopyranoside (MMP), 10 mM NaPO₄, 80 mM NaCl, 0.1% Triton X-100, pH 6.8. The elution peak, about 1 column volume, was collected, 02 μm filtered and stored at or below −60° C. for HAP chromatography.

HAP chromatography was conducted at room temperature. A 1200 ml (100×150 mm) type I ceramic hydroxyapatite column (BioRad) was conditioned with one column volume of 0.4 M NaPO₄, pH 6.8, then equilibrated with not less than ten column volumes of 10 mM NaPO₄, 80 mM NaCl, 0.1% Triton X-100, pH 6.8. Four lots of GNA eluate pools were thawed in a circulating water bath at not more than 30° C., 0.2 μm filtered and loaded onto the equilibrated column at 131 ml/min (100 cm/hr). HAP equilibration buffer was applied to the column as a chase buffer following the load. The flow-through was collected when UV rose above baseline. The product collection was stopped when the product pool volume reached to a volume of load volume plus 75% of the column volume. The HAP flow-through pool was further processed by DV50 viral reduction filtration.

DV50 Filtration was conducted at room temperature. DV50 load was prepared by diluting the HAP pool two-fold and adjusting to 0.15% Triton X-100, 1 mM EDTA, pH 5.3. Dilution and adjustment were achieved by adding Dilution Buffer-1 (3 mM citric acid, 2 mM EDTA, 0.2% Triton X-100) to adjust the pH of the product pool to 5.3, followed by addition of Dilution Buffer-2 (2 mM EDTA, 0.2% Triton X-100, pH 5.3) to bring the final volume to 2-fold of the original HAP pool volume.

The diluted and adjusted HAP pool (DV50 Load) was filtered through a 10-inch, Pall Ultipor VF DV50 membrane cartridge (Pall). The filter housing was assembled with filter cartridge, prewetted with water, and sterilized by autoclaving at 123° C. for 60 minutes with slow exhaust prior to use. The filter was then prewetted with SP equilibration buffer (10 mM Sodium Citrate, 1 mM EDTA, 0.15% Triton X-100, pH 5.3), and drained before application of the DV50 load at a pressure not more than 45 psi. DV50 load was subsequently applied with a flux rate of about 800 ml/min at a transmembrane pressure of about 30 psi. The filtrate was collected and stored at 2-8° C. overnight and used in the SP step.

SP chromatography was conducted at room temperature in room. An 88-ml (50×45 mm) SP Sepharose HP column (Pharmacia, Peapack, N.J.) was equilibrated with 15 column volumes of equilibration buffer (10 mM Sodium Citrate, 1 mM EDTA, 0.15% Triton X-100, pH 5.3). The DV50 filtrate was applied to the column. The column was washed first with 5 column volumes of equilibration buffer followed by 20 column volumes of wash buffer containing 10 mM Sodium Citrate, 15 mM NaCl, 1 mM EDTA, 0.1% Tween-80™, pH 6.0. Product was eluted from the column with 10 mM Sodium Citrate, 180 mM NaCl, 1 mM EDTA, 0.1% Tween-80™, pH 6.0. The entire 280 nm absorption peak was collected as product pool. The product pool was stored at 2-8° C. overnight and used in the Q-membrane filtration step.

The Q-membrane filtration step was conducted at room temperature. Two sterilized Sartorious Q100× disc membranes were connected in series. The membranes were equilibrated with not less than 300 ml of Q equilibration buffer (10 mM Sodium Citrate, 180 mM NaCl, 1 mM EDTA, 0.1% Tween-80™, pH 6.0). The entire SP eluate pool was filtered through equilibrated Q membranes at a flow rate of 30-100 ml/min, followed by flushing with 40 ml of Q equilibration buffer. The filtrate and the flush were collected and combined as the product pool and used in the G25 step.

The G25 step was conducted at room temperature. A 1115-ml (100×142 mm) Pharmacia Sephadex G-25 column (Pharmacia, Peapack, N.J.) was equilibrated with not less than five column volumes of formulation buffer (10 mM Sodium Citrate, 270 mM NaCl, 1 mM EDTA, 0.1% Tween-80™, pH 6.0). Q filtrate pool was applied to the column and the column flow-through collected, filtered through a 0.22 μm filter (Millipore) and stored frozen at −60° C. or below, until use.

### EXAMPLE 3

### Immunogenicity of HCV E1E2 Vaccine Compositions in Mice

The immunogenicity of HCV E1E2₈₀₉, produced and purified as described above, in combination with a submicron oil-in-water emulsion and/or a CpG oligonucleotide, was determined as follows.

The formulations used in this study are summarized in Table 1. MF59, a submicron oil-in-water emulsion which contains 4-5% w/v squalene, 0.5% w/v Tween 80™, 0.5% Span 85™, was produced as described previously. See, International Publication No. WO 90/14837; U.S. Pat. No. 6,299,884, incorporated herein by reference in its entirety; and Ott et al., “MF59—Design and Evaluation of a Safe and Potent Adjuvant for Human Vaccines” in Vaccine Design: The Subunit and Adjuvant Approach (Powell, M. F. and Newman, M. J. eds.) Plenum Press, New York, 1995, pp. 277-296. For groups 4 and 9, four times the amount of MF59 was used. The MF59 used in this study was MF59-0, and did not contain any MTP-PE.

The formulations used for groups 1, 3, 6 and 8 also included 25 μg of an active CpG molecule per dose. The sequence of the active CpG molecule used was: 5′-TCCATGACGTTCCTGACGTT-3′ (SEQ ID NO:1).

The formulation used for group 5 included 25 μg of an inactive control CpG molecule per dose. The sequence of the inactive CpG molecule used was: 5′-TCCAGGACTTCTCTCAGGTT-3′ (SEQ ID NO:2).

The formulations used for groups 1-4 included 2.8 μg per dose of the HCV E1E2₈₀₉ antigen, produced as described above.

The formulations used for groups 5-9 included 2.0 μg per dose of HCV E2₇₁₅, a truncated E2 protein, produced in CHO cells, as described in U.S. Pat. No. 6,12,020.

Balb/C mice, six weeks of age, were divided into 9 groups (10 mice per group) and administered, intramuscularly 50 μl of a vaccine composition with the components specified in Table 1. Animals were boosted at 30 and 90 days following the initial injection. Serum was collected 14 days following the last injection and anti-E1E2 and anti-E2 antibody titers determined by enzyme immunoassays. See, Chien et al., Lancet (1993) 342:933.

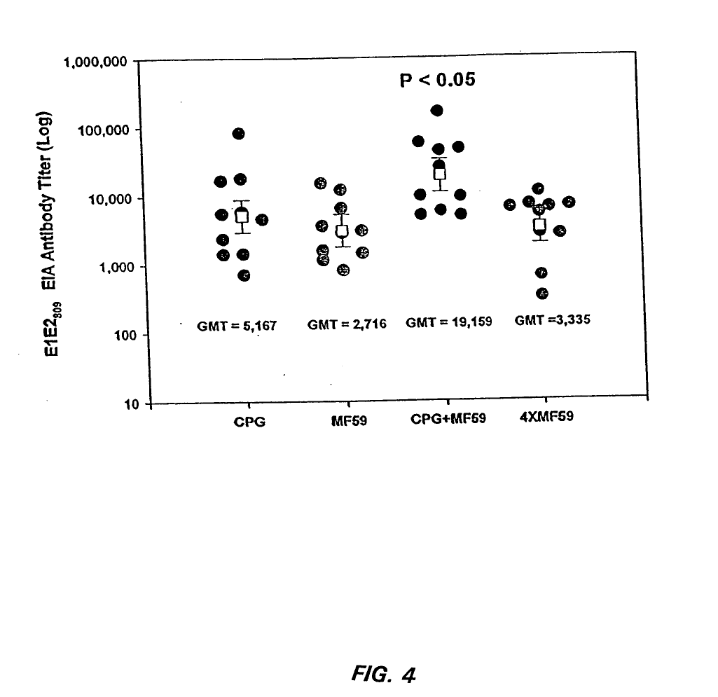

The results are shown in Table 1 and FIG. 4. As can be seen, mice immunized with HCV E1E2 using CpG combined with MF59 as adjuvant, produced significantly higher (P<0.05) levels of E1E2 antibodies than mice immunized with E1E2 using MF59 alone or 4xMF59 alone as adjuvants. CpG alone produced antibody levels higher than antibody levels with MF59 alone, albeit, not significantly higher. In contrast, mice immunized with E2₇₁₅ using MF59 and/or CpG, produced very low levels of antibodies with less than 50% of the mice responding. This is surprising as previous experiments with E2₇₁₅ have produced high antibody levels in mice, with all mice tested responding.

TABLE 1
Immunogenicity of HCV E1E2₈₀₉ and E2₇₁₅ using CPG and or MF59 as
adjuvants. The numbers in parenthesis indicate the number of animals
producing antibodies relative to the number of animals immunized.

| | | | Geometric | |
|---|---|---|---|---|
| | | | Mean E1E2 | Geometric |
| | Vaccine; | | EIA Antibody | Mean E2 EIA |
| Group | Adjuvant | Dose | Titer | Antibody Titer |
| 1 | E1E2₈₀₉; | 2.8, 2.8, 2.8 | 5,167 | ND |
| | CpG | | (10/10) | |
| 2 | E1E2₈₀₉; | 2.8, 2.8, 2.8 | 2,716 | ND |
| | MF59 | | (10/10) | |
| 3 | E1E2₈₀₉; | 2.8, 2.8, 2.8 | 19,159^{B} | ND |
| | CpG + MF59 | | (10/10) | |
| | | | P < 0.05 | |
| 4 | E1E2₈₀₉; | 2.8, 2.8, 2.8 | 3,335 | ND |
| | 4X MF59 | | (10/10) | |
| 5 | E2₇₁₅; | 2.0, 2.0, 2.0 | ND | 1.3 |
| | Control CpG | | | (1/10) |
| 6 | E2₇₁₅; | 2.0, 2.0, 2.0 | ND | 3.1 |
| | CpG | | | (2/20) |
| 7 | E2₇₁₅; | 2.0, 2.0, 2.0 | ND | 6.1 |
| | MF59 | | | (4/10) |
| 8 | E2₇₁₅; | 2.0, 2.0, 2.0 | ND | 26.8 |
| | CpG + MF59 | | | (5/10) |
| 9 | E2₇₁₅; | 2.0, 2.0, 2.0 | ND | 9.7 |
| | 4xMF59 | | | (4/10) |

### EXAMPLE 4

### Immunogenicity of HCV E1E2 Vaccine Compositions in Chimpanzees

The immunogenicity of HCV E1E2₈₀₉, produced and purified as described above, in combination with a submicron oil-in-water emulsion and/or a CpG oligonucleotide, was determined as follows.

The formulations used in this study are summarized in Table 2. MF59 and E1E2₈₀₉ are described above. The sequence of the CpG molecule used was: 5′-TCGTCGTTTTGTCGTTTTGTCGTT-3′ (SEQ ID NO:5).

Chimpanzees were divided into 2 groups (5 animals per group) and administered, intramuscularly a vaccine composition with the components specified in Table 1. In particular, one group of animals was immunized at 0, 1 and 6 months with 20 μg of E1E2₈₀₉ and MF59. The second group of animals was also immunized at 0, 1 and 6 months with 20 fig of E1E2₈₀₉ and MF59, as well as with 500 μg CpG.

Serum samples were obtained 14 days after the last immunization and anti-E1E2 antibody titers determined by enzyme immunoassays. In particular, the E1E2 antigen was coated on polystyrene microtiter plates and bound antibody was detected with a HRP-conjugated anti-human antibody followed by tetramethylbenzidine substrate development.

As can be seen in Table 2, chimpanzees immunized with HCV E1E2 using CpG combined with MF59 as adjuvant, produced significantly higher (P<0.05) levels of E1E2 antibodies than animals immunized with E1E2 using MF59 alone.

TABLE 2
Immunogenicity of HCV E1E2₈₀₉ using CPG and MF59 as adjuvants.

| | | | Geometric |
|---|---|---|---|
| | | | Mean E1E2 |
| Vaccine; | | E1E2 EIA | EIA Antibody |
| Adjuvant | Chimp | Antibody Titer | Titer |
| Group 1: | 1 | 84 | 261 |
| E1E2₈₀₉; | 2 | 101 | |
| CpG | 3 | 131 | |
| | 4 | 421 | |
| | 5 | 2580 | |
| Group 2: | 1 | 8835 | 2713 |
| E1E2₈₀₉; | 2 | 2713 | |
| CpG + MF59 | 3 | 3201 | |
| | 4 | 510 | |
| | 5 | 1238 | |

Accordingly, novel HCV vaccine compositions and methods of using the same are disclosed. From the foregoing, it will be appreciated that, although preferred embodiments of the subject invention have been described in some detail, it is understood that obvious variations can be made without departing from the spirit and the scope of the invention as defined by the appended claims.

1. A composition comprising:
(a) a hepatitis C virus (HCV) envelope antigen;
(b) a submicron oil-in-water emulsion that lacks N-acetylmuramyl-L-alanyl-D-isogluatminyl-L-alanine-2-(1′-2′-dipalmitoyl-sn-glycero-3-huydroxyphosphoryloxy)-ethylamine (MTP-PE); and
(c) an immunostimulatory nucleic acid sequence (ISS).

2. The composition of claim 1, wherein the HCV envelope antigen comprises HCV E1E2 complexes.

3. The composition of claim 2, wherein at least one of the HCV E1E2 complexes consists of:
(a) amino acids 20-210 of SEQ ID NO:4 or sequence of amino acids with at least 80% sequence identity to the contiguous sequence of amino acids 20-210 of SEQ ID NO:4;
(b) amino acids 211-574 of SEQ ID NO:4 or sequence of amino acids with at least 80% sequence identity to the contiguous sequence of amino acids 211-574 of SEQ ID NO:4; and
(c) amino acids 575-637 of SEQ ID NO:4 or sequence of amino acids with at least 80% sequence identity to the contiguous sequence of amino acids 575-637 of SEQ ID No:4.

4. The composition of claim 3, wherein at least one of the HCV E1E2 antigen complexes consists of:
(a) amino acids 20-210 of SEQ ID NO:4;
(b) amino acids 211-574 of SEQ ID NO:4; and
(c) amino acids 575-637 of SEQ ID NO:4.

5. The composition of claim 1, wherein the ISS is a CpG oligonucleotide.

6. The composition of claim 5, wherein the CpG oligonucleotide comprises the sequence 5′-X₁X₂CGX₃×4, where X₁ and X₂ are a sequence selected from the group consisting of GpT, GpG, GpA, ApA, ApT, ApG, CpT, CpA, CpG, TpA, TpT and TpG; and X₃ and X₄ are selected from the group consisting of TpT, CpT, ApT, ApG, CpG, TpC, ApC, CpC, TpA, ApA, GpT, CpA, and TpG, wherein p signifies a phosphate bond.

7. The composition of claim 5, wherein the CpG oligonucleotide comprises the sequence GACGTT, GACGTC, GTCGTT or GTCGCT.

8. The composition of claim 7, wherein the CpG oligonucleotide comprises the sequence 5′-TCCATGACGTTCCTGACGTT-3′(SEQ ID NO:1).

9. The composition of claim 7, wherein the CpG oligonucleotide comprises the sequence 5′-TCGTCGTTTTGTCGTTTTGTCGTT-3′(SEQ ID NO:5).

10. The composition of claim 1, wherein the submicron oil-in-water emulsion comprises:
(1) a metabolizable oil, wherein the oil is present in an amount of 0.5% to 20% of the total volume; and
(2) an emulsifying agent, wherein the emulsifying agent is present in an amount of 0.01% to 2.5% by weight (w/v), and wherein the oil and the emulsifying agent are present in the form of an oil-in-water emulsion having oil droplets substantially all of which are about 100 nm to less than 1 micron in diameter.

11. The composition of claim 10, wherein the oil is present in an amount of 1% to 12% of the total volume and the emulsifying agent is present in an amount of 0.01% to 1% by weight (w/v).

12. The composition of claim 10, wherein the emulsifying agent comprises a polyoxyethylene sorbitan mono-, di-, or triester and/or a sorbitan mono-, di-, or triester.

13. The composition of claim 10, wherein the submicron oil-in-water emulsion comprises 4-5% w/v squalene, 0.25-1.0% w/v polyoxyelthylenesorbitan monooleate, and/or 0.25-1.0% sorbitan trioleate.

14. The composition of claim 13, wherein the submicron oil-in-water emulsion consists essentially of 5% by volume of squalene; and one or more emulsifying agents selected from the group consisting of polyoxyelthylenesorbitan monooleate and sorbitan trioleate, wherein the total amount of emulsifying agent(s) present is 1% by weight (w/v).

15. The composition of claim 14, wherein the one or more emulsifying agents are polyoxyelthylenesorbitan monooleate and sorbitan trioleate and the total amount of polyoxyelthylenesorbitan monooleate and sorbitan trioleate present is 1% by weight (w/v).

16. A method of stimulating an immune response in a vertebrate subject which comprises administering to the subject a therapeutically effective amount of a hepatitis C virus (HCV) envelope antigen, a submicron oil-in-water emulsion that lacks N-acetylmuramyl-L-alanyl-D-isogluatminyl-L-alanine-2-(1′-2′-dipalmitoyl-sn-glycero-3-huydroxyphosphoryloxy)-ethylamine (MTP-PE), and an immunostimulatory nucleic acid sequence (ISS).

17. The method of claim 16, wherein the HCV envelope antigen comprises HCV E1E2 complexes.

18. The method of claim 17, wherein at least one of the HCV E1E2 complexes consists of:
(a) amino acids 20-210 of SEQ ID NO:4 or sequence of amino acids with at least 80% sequence identity to the contiguous sequence of amino acids 20-210 of SEQ ID NO:4;
(b) amino acids 211-574 of SEQ ID NO:4 or sequence of amino acids with at least 80% sequence identity to the contiguous sequence of amino acids 211-574 of SEQ ID NO:4; and
(c) amino acids 575-637 of SEQ ID NO:4 or sequence of amino acids with at least 80% sequence identity to the contiguous sequence of amino acids 575-637 of SEQ ID NO:4.

19. The method of claim 18, wherein at least one of the HCV E1 E2 antigen complexes consists of:
(a) amino acids 20-210 of SEQ ID NO:4;
(b) amino acids 211-574 of SEQ ID NO:4; and
(c) amino acids 575-637 of SEQ ID NO:4.

20. The method of claim 16, wherein the submicron oil-in-water emulsion is present in the same composition as the antigen.

21. The method of claim 16, wherein the ISS is a CpG oligonucleotide.

22. The method of claim 21, wherein the CpG oligonucleotide comprises the sequence 5′-X₁X₂CGX₃×4, where X₁ and X₂ are a sequence selected from the group consisting of GpT, GpG, GpA, ApA, ApT, ApG, CpT, CpA, CpG, TpA, TpT and TpG; and X₃ and X₄ are selected from the group consisting of TpT, CpT, ApT, ApG, CpG, TpC, ApC, CpC, TpA, ApA, GpT, CpA, and TpG, wherein p signifies a phosphate bond.

23. The method of claim 21, wherein the CpG oligonucleotide comprises the sequence GACGTT, GACGTC, GTCGTT or GTCGCT.

24. The method of claim 23, wherein the CpG oligonucleotide comprises the sequence 5′-TCCATGACGTTCCTGACGTT-3′ (SEQ ID NO:1).

25. The method of claim 23, wherein the CpG oligonucleotide comprises the sequence 5′-TCGTCGTTTTGTCGTTTTGTCGTT-3′ (SEQ ID NO:5).

26. The method of claim 16, wherein the submicron oil-in-water emulsion comprises:
(1) a metabolizable oil, wherein the oil is present in an amount of 0.5% to 20% of the total volume; and
(2) an emulsifying agent, wherein the emulsifying agent is present in an amount of 0.01% to 2.5% by weight (w/v), and wherein the oil and the emulsifying agent are present in the form of an oil-in-water emulsion having oil droplets substantially all of which are about 100 nm to less than 1 micron in diameter.

27. The method of claim 26, wherein the oil is present in an amount of 1% to 12% of the total volume and the emulsifying agent is present in an amount of 0.01% to 1% by weight (w/v).

28. The method of claim 26, wherein the emulsifying agent comprises a polyoxyethylene sorbitan mono-, di-, or triester and/or a sorbitan mono-, di-, or triester.

29. The method of claim 28, wherein the submicron oil-in-water emulsion comprises 4-5% w/v squalene, 0.25-1.0% w/v polyoxyelthylenesorbitan monooleate, and/or 0.25-1.0% sorbitan trioleate.

30. The method of claim 29, wherein the submicron oil-in-water emulsion consists essentially of 5% by volume of squalene; and one or more emulsifying agents selected from the group consisting of polyoxyelthylenesorbitan monooleate and sorbitan trioleate, wherein the total amount of emulsifying agent(s) present is 1% by weight (w/v).

31. The method of claim 30, wherein the one or more emulsifying agents are polyoxyelthylenesorbitan monooleate and sorbitan trioleate and the total amount of polyoxyelthylenesorbitan monooleate and sorbitan trioleate present is 1% by weight (w/v).

32. A method of making a composition comprising combining a submicron oil-in-water emulsion that lacks N-acetylmuramyl-L-alanyl-D-isogluatminyl-L-alanine-2-(1′-2′-dipalmitoyl-sn-glycero-3-huydroxyphosphoryloxy)-ethylamine (MTP-PE), with a hepatitis C virus (HCV) envelope antigen and an immunostimulatory nucleic acid sequence (ISS).
